(19) United States
(12) Patent Application Publication
(10) Pub. No.: US 2004/0223987 A1
(43) Pub. Date: Nov. 11, 2004

(54) Composition structured with a polymer containing a heteroatom and organogelator

(76) Inventor: Veronique Ferrari, Maisons-Alfort (FR)

Correspondence Address: FINNEGAN, HENDERSON, FARABOW, GARRETT & DUNNER, LLP, 1300 I STREET, NW, WASHINGTON, DC, 20005, US

(21) Appl. No.: 10/129,377

(22) PCT Filed: Dec. 12, 2001
(86) PCT No.: PCT/IB01/02780

(30) Foreign Application Priority Data
Dec. 13, 2000 (WO) PCT/IB00/02028

(51) Int. Cl. A61K 7/00

(52) U.S. Cl. 424/401

(57) ABSTRACT

A physiologically acceptable composition, in particular a cosmetic composition, comprising at least one liquid fatty phase which comprises (i) at least one structuring polymer having a polymer skeleton which comprises at least one hydrocarbon-based repeating unit comprising at least one hetero atom; and (ii) at least one organogelator.

The present invention relates to a care and/or treatment and/or make-up composition for the skin, including the scalp, and/or for the lips of human beings, and/or for other keratin materials, such as keratinous fibers, containing a liquid fatty phase, structured with a specific polymer containing a hetero atom. This composition is stable over time and can be used in the form of a rod or stick of make-up and more especially of lipstick, the application of which produces a glossy deposit with good staying power or long-wearing properties.

It is common to find a structured, i.e., gelled and/or rigidified, liquid fatty phase in cosmetic or dermatological products; this is especially the case in solid compositions such as deodorants, lip balms, lipsticks, concealer products, eyeshadows and cast foundations. This structuring may be obtained with the aid of waxes and/or fillers. Unfortunately, these waxes and fillers may have a tendency to make the composition matte, which may not always be desirable, in particular for a lipstick or an eyeshadow; specifically, consumers are always on the lookout for a lipstick in stick form which can deposit a film with good staying power or long wearing properties but which is also increasingly glossy.

The structuring of the liquid fatty phase may make it possible in particular to limit its exudation (or syneresis) from solid compositions, particularly in hot and humid areas and, furthermore, after deposition on the skin or the lips, to limit the migration of this phase into wrinkles and fine lines, a characteristic particularly desirable in a lipstick or eyeshadow. The reason for this is that considerable migration of the liquid fatty phase, particularly when it is charged with coloring agents, may lead to an unpleasant appearance around the lips and the eyes, making wrinkles and fine lines particularly prominent. Consumers often state this migration as being a major drawback of conventional lipsticks. The term “migration” means movement of the composition beyond its initial site of application.

Gloss of a lipstick or other cosmetic is generally associated with the nature of the liquid fatty phase. Thus, it may be possible to reduce the amount of waxes and/or fillers in the composition in order to increase the gloss of a lipstick or cosmetic, but in that case the migration of the liquid fatty phase may increase. In other words, the amounts of waxes and of fillers required to prepare a stick of suitable hardness which does not exude at room temperature are a restricting factor on the gloss of the deposit.

To overcome at least one (as used throughout herein, the expression “at least one” means one or more and thus includes individual components as well as mixtures/combinations) of these drawbacks, the inventors have envisaged replacing all or some of the waxes and/or fillers with polymers for structuring the liquid fatty phase, for example, polyamide polymers. However, the cosmetic sticks obtained may be of insufficient mechanical strength when applied to the lips or the skin. This mechanical fragility may be reflected by breakage of the stick during its shear on application.

Document JP-A-10-090110 discloses transparent cosmetic compositions based on polyamide combined with a pentaerythritol ester and colophony and with partial esters of branched or unsaturated fatty acids and of polyglycerol. This combination makes it possible to reinforce the texture of the compositions in stick form and also their stability. However, this composition contains ingredients such as colophony which is an allergenic product and which entails the risk of sensitizing individuals with reactive and/or hypersensitive skin. The use of this composition is thus limited, all the more so since more than 50% of human beings have skin that is reactive and/or sensitive to cosmetic ingredients.

Furthermore, make-up compositions should have good staying power or long-wearing properties over time, i.e., little turning of or change in color over time or a gradual or homogeneous change of the deposit over time. The turning of or change in color of the deposit may be due, for lipsticks, to an interaction with saliva and, for foundations and eyeshadows, to an interaction with the sweat and sebum secreted by the skin. Specifically, a composition which has no staying power or long wearing properties may oblige the user to reapply make-up regularly. However, consumers nowadays wish to enhance the beauty of their face or body while spending as little time as possible in doing so.

The need thus remains for a composition which does not have the above drawbacks, which especially has good stability over time, even in hot and humid countries, and which produces a deposit on the skin or the lips that has at least one of the following properties: good staying power or long-wearing properties over time, non-migrating properties, a glossy appearance, and able to withstand shear during application. Furthermore, this composition should be easy to manufacture and should comprise no sensitizing ingredients and/or ingredients giving rise to allergies and/or itching on the skin or the lips.

Accordingly, the present invention is drawn to a care and/or make-up and/or treatment composition for the skin and/or the lips of the face and/or for superficial body growths, and/or for keratinous materials, such as keratinous fibers, such as hair, which may make it possible to overcome at least one of the drawbacks mentioned above. It is to be noted that a deodorant product is a body hygiene product and does not relate to care, make-up or treatment of keratin materials, including keratinous fibers, skin, or lips.

The inventors have found, surprisingly, that the use of specific polymers combined with at least one molecular organic agent able to gel the liquid fatty phase, i.e., an organogelator, may make it possible to obtain a stick which can be mechanically strong even during application to the lips or the skin, and whose application can produce a deposit which has noteworthy cosmetic properties. For example, the deposit is at least one of glossy, supple, comfortable and “migration-resistant”. Furthermore, the composition may be stable over time and may not exude at room temperature. In addition, the structuring of the fatty phase of the composition may produce a product that is easy to handle since it does not run between the fingers, unlike a liquid product.

The term “stable” means a composition which does not exude at room temperature (25° C.) for at least 2 months, such as, for example, at least 9 months.

The invention applies not only to make-up products for the lips, such as lipsticks, lip glosses and lip pencils, but also to care and/or treatment products for the skin, including the scalp, and for the lips, such as antisun products, especially in stick form for facial skin or the lips, care products for the human face or body, make-up products for the skin, both of the human face and body, such as foundations optionally cast in stick or dish form, concealer products, eyeshadows, face powders, transfer tattoos, body hygiene products such as deodorants especially in stick form, shampoos, conditioners and make-up products for the eyes such as eyeliners, eye pencils and mascaras more especially in cake form, as well as make-up and care products for superficial body growths, for instance keratinous fibres or nails. As used herein, “keratinous fibres” means hair on the head, the eyelashes and the eyebrows.

More specifically, the present invention is drawn to a structured composition containing at least one liquid fatty phase structured with at least one structuring polymer comprising a polymer skeleton comprising at least one hydrocarbon-based repeating unit comprising at least one hetero atom. In one embodiment, the at least one structuring polymer further comprises at least one chain chosen from:

(i) terminal fatty chains, optionally functionalized, chosen from alkyl chains, such as alkyl chains comprising at least four carbon atoms, and alkenyl chains, such as alkenyl chains comprising at least four carbon atoms, bonded to the polymer skeleton, such as a polyamide skeleton, via at least one linking group, and

(ii) pendant fatty chains, optionally functionalized, chosen from alkyl chains, such as alkyl chains comprising at least four carbon atoms, and alkenyl chains, such as alkenyl chains comprising at least four carbon atoms, bonded to the polymer skeleton, such as a polyamide skeleton, via at least one linking group.

A linking group, for example, may be chosen from a direct bond, ureas, urethanes, thiourethanes, thioesters, thioethers, thioureas, esters, ethers, amines, and combinations thereof. In one embodiment, the linking group is chosen from ureas, esters, and amines. As a further example, the linking group is chosen from esters and amines.

In another embodiment, at least one of the terminal fatty chains and the pendant chains, optionally functionalized, comprises from 8 to 120 carbon atoms, such as in the form of alkyl and alkenyl chains, bonded to the polymer skeleton via at least one linking group. As is evident, the at least one structuring polymer may comprise both at least one pendant fatty chain and at least one terminal fatty chain as defined above.

The liquid fatty phase further contains at least one organogelator able to gel the liquid fatty phase. The at least one liquid fatty phase, the at least one structuring polymer and the at least one molecular organogelator together form a physiologically acceptable medium.

In one embodiment, the at least one structuring polymer has a low weight-average molecular mass, such as less than 100,000. However, this weight-average molecular mass can be present up to 500 000 and even up to 1 000 000.

The composition of the invention can be in the form of a paste, a solid or a more or less viscous cream. It can be a rigid or soft single or multiple emulsion, such as an oil-in-water or water-in-oil emulsion or an oil-in-water-in-oil emulsion or water-in-oil-in-water emulsion, or a rigid or soft gel containing an oily continuous phase. For example, the liquid fatty phase can be the continuous phase of the composition. In one embodiment, the composition is anhydrous. In another embodiment, the composition is in a form cast as a stick or in a dish, for example, solid, and further example, in the form of an oily rigid gel, such as an anhydrous gel, e.g., an anhydrous stick. In a further embodiment, the composition is in the form of an opaque or translucent rigid gel (depending on the presence or absence of pigments), and in a specific example, the liquid fatty phase forms the continuous phase. In one embodiment, the composition is chosen from molded and poured sticks.

The organogelator of the invention, which is a non-polymeric organic gelling agent, may make it possible to reinforce the mechanical properties of the composition, for example of the composition in stick form. This reinforcement can be demonstrated by a stick which withstands the shear generated during the application of the composition to the lips or the skin, as well as to superficial body growths, such as keratinous matters or fibers. Thus, it is possible to manufacture a stick of lipstick with a stick diameter of 12.7 mm, this diameter corresponding to that usually used in conventional lipsticks.

For the purposes of the invention, the expression “liquid fatty phase” means a fatty phase which is liquid at room temperature (25° C.) and atmospheric pressure (760 mmHg, i.e. 101 KPa), and comprises at least one fatty substance that is liquid at room temperature, also referred to as an oil. This fatty phase may comprise several fatty substances that are generally mutually compatible, i.e forming a homogeneous phase macroscopically. The expression “liquid fatty substance” means a non-aqueous liquid medium which is immiscible in all proportions with water, for example, a hydrocarbon-based compound comprising at least one carbon chain containing at least 5 carbon atoms and possibly comprising at least one polar group chosen from carboxylic acid, hydroxyl, polyol, amine, amide, phosphoric acid, phosphate, ester, ether, urea, carbamate, thiol, thioether and thioester; a silicone compound optionally comprising carbon chains at the end or pendant, these chains optionally being substituted with a group chosen from fluoro, perfluoro, (poly)amino acid, ether, hydroxyl, amine, acid and ester groups; or a fluoro or perfluoro compound such as fluorohydrocarbons or perfluorohydrocarbons containing at least 5 carbon atoms, possibly comprising a hetero atom chosen from N, O, S and P and optionally at least one polar function chosen from ether, ester, amine, acid, carbamate, urea, thiol and hydroxyl groups. In practice, the total liquid fatty phase may be present, for example, in an amount ranging from 1% to 99% by weight relative to the total weight of the composition; for example from 5 to 99%, further examples include ranges of 5% to 95.5%, 10% to 80%, and 20% to 75%.

The structuring of the liquid fatty phase can be modified according to the nature of the polymer containing a. hetero atom and of the organogelator used, and may be such that a rigid structure in the form of a rod or stick with good mechanical strength is obtained. When these rods or sticks are colored, they may make it possible, after application, to obtain a uniformly colored glossy deposit which does not migrate and which has good staying power or long-wearing properties, in particular of the color, over time. The composition may contain at least one structuring polymer and at least one organogelator.

In one embodiment, the composition of the invention is a composition for the lips such as a lipstick composition, e.g., in stick form.

Structuring Polymer

In one embodiment, the at least one structuring polymer in the composition of the invention is a solid that is not deformable at room temperature (25° C.) and atmospheric pressure (760 mmHg, i.e. 101 KPa). In a further embodiment, the at least one structuring polymer is capable of structuring the composition without opacifying it. The inventors think that is due to the fact that the polymer does not crystallize. Moreover, the structuration of the liquid phase is due to hydrogen interactions between two molecules of polymer and/or between the polymer and the liquid fatty phase. As defined above, the at least one structuring polymer of the present invention comprises a polymer skeleton comprising at least one hydrocarbon-based repeating unit comprising at least one hetero atom. In one embodiment, the at least one structuring polymer further comprises at least one terminal fatty chain chosen from alkyl and alkenyl chains, such as of at least 4 atoms, and further such as comprising 8 to 120 carbon atoms, bonded to the polymer skeleton via at least one linking group. The terminal fatty chain may, for example, be functionalized. The at least one structuring polymer may also further comprise at least one pendant fatty chain chosen from alkyl and alkenyl chains, such as of at least 4 atoms, and further such as comprising 8 to 120 carbon atoms, bonded to any carbon or hetero atom of the polymer skeleton via at least one linking group. The pendant fatty chain may, for example, be functionalized. The at least one structuring polymer may comprise both at least one pendant fatty chain and at least one terminal fatty chain as defined above, and one or both types of chains can be functionalized.

In one embodiment, the structuring polymer comprises at least two hydrocarbon-based repeating units. As a further example, the structuring polymer comprises at least three hydrocarbon-based repeating units and as an even further example, the at least three repeating units are identical.

As used herein, “functionalized” means comprising at least one functional group. Non-limiting examples of functional groups include hydroxyl groups, ether groups, oxyalkylene groups, polyoxyalkylene groups, carboxylic acid groups, amine groups, amide groups, halogen containing groups, including fluoro and perfluoro groups, halogen atoms, ester groups, siloxane groups and polysiloxane groups.

For purposes of the invention, the expression “functionalized chain” means, for example, an alkyl chain comprising at least one functional (reactive) group chosen, for example, from those recited above. For example, in one embodiment, the hydrogen atoms of at least one alkyl chain may be substituted at least partially with fluorine atoms.

According to the invention, these chains may be linked directly to the polymer skeleton or via an ester function or a perfluoro group.

For the purposes of the invention, the term “polymer” means a compound containing at least 2 repeating units, such as, for example, a compound containing at least 3 repeating units, which may be identical.

As used herein, the expression “hydrocarbon-based repeating unit” includes a repeating unit comprising from 2 to 80 carbon atoms, such as, for example, from 2 to 60 carbon atoms. The at least one hydrocarbon-based repeating unit may also comprise oxygen atoms. The hydrocarbon-based repeating unit may be chosen from saturated and unsaturated hydrocarbon-based repeating units which in turn may be chosen from linear hydrocarbon-based repeating units, branched hydrocarbon-based repeating units and cyclic hydrocarbon-based repeating units. The at least one hydrocarbon-based repeating unit may comprise, for example, at least one hetero atom that is part of the polymer skeleton, i.e., not pendant. The at least one hetero atom may be chosen, for example, from nitrogen, sulphur, and phosphorus. For example, the at least one hetero atom may be a nitrogen atom, such as a non-pendant nitrogen atom. In another embodiment, the at least one hydrocarbon-based repeating unit may comprise at least one hetero atom with the proviso that the at least one hetero atom is not nitrogen. In another embodiment, the at least one hetero atom is combined with at least one atom chosen from oxygen and carbon to form a hetero atom group. In one embodiment, the hetero atom group comprises a carbonyl group.

The at least one unit repeating comprising at least one hetero atom may be chosen, for example, from amide groups, carbamate groups, and urea groups. In one embodiment, the at least one repeating unit comprises amide groups forming a polyamide skeleton. In another embodiment, the at least one repeating unit comprises carbamate groups and/or urea groups forming a polyurethane skeleton, a polyurea skeleton and/or a polyurethane-polyurea skeleton. The pendant chains, for example, can be linked directly to at least one of the hetero atoms of the polymer skeleton. In yet another embodiment, the at least one hydrocarbon-based repeating unit may comprise at least one hetero atom group with the proviso that the at least one hetero atom group is not an amide group. In one embodiment, the polymer skeleton comprises at least one repeating unit chosen from silicone units and oxyalkylene units, the at least one repeating unit being between the hydrocarbon-based repeating units.

In one embodiment, the composition of the invention comprises at least one structuring polymer with nitrogen atoms, such as amide, urea or carbamate units, and preferably amide units, and at least one polar oil.

In one embodiment, in the at least one structuring polymer, the percentage of the total number of fatty chains ranges from 40% to 98% relative to the total number of repeating units and fatty chains, and as a further example, from 50% to 95%. In a further embodiment wherein the polymer skeleton is a polyamide skeleton, in the at least one structuring polymer, the percentage of the total number of fatty chains ranges from 40% to 98% relative to the total number of all amide units and fatty chains, and as a further example, from 50% to 95%.

In a further embodiment, the nature and proportion of the at least one hydrocarbon-based repeating unit comprising at least one hetero atom depends on the nature of a liquid fatty phase of the composition and is, for example, similar to the nature of the fatty phase. For example, not to be limited as to theory, the more polar the hydrocarbon-based repeating units containing a hetero atom, and in high proportion, which corresponds to the presence of several hetero atoms, the greater the affinity of the at least one structuring polymer to polar oils. Conversely, the more non-polar, or even apolar, and lesser in proportion the hydrocarbon-based repeating units containing a hetero atom, the greater the affinity of the polymer for apolar oils.

In another embodiment, the invention is drawn to a structured composition containing at least one liquid fatty phase structured with at least one structuring polymer, wherein said at least one structuring polymer is a polyamide comprising a polymer skeleton comprising at least one amide repeating unit and optionally at least one pendant fatty chain and/or at least one terminal chain that are optionally functionalized and comprise from 8 to 120 carbon atoms, bonded to at least one of the amide repeating units via at least one linking group. The liquid fatty phase further contains at least one organogelator able to gel the liquid fatty phase. The at least one liquid fatty phase, the at least one structuring polyamide and the at least one organogelator together form a physiologically acceptable medium.

When the structuring polymer has amide repeating units, the pendant fatty chains may be linked to at least one of the nitrogen atoms in the amide repeating units.

The structuring polymer, for example the polyamide polymer, may have a weight-average molecular mass of less than 100,000, such as less than 50,000. In another embodiment, the weight-average molecular mass may range from 1000 to 30,000, such as from 2000 to 20,000, further such as from 2000 to 10,000.

The structuring polymer, as for example the polyamide polymer, is non soluble in water or in an aqueous phase. In another embodiment the structuring polymer has non ionic group.

As discussed, the at least one structuring polymer may, for example, be chosen from polyamide polymers. A polyamide polymer may comprise, for example, a polymer skeleton which comprises at least one amide repeating unit, i.e., a polyamide skeleton. In one embodiment, the polyamide skeleton may further comprise at least one terminal fatty chain chosen from alkyl chains, for example, alkyl chains comprising at least four carbon atoms, and alkenyl chains, for example, alkenyl chains comprising at least four carbon atoms, bonded to the at least one polyamide skeleton via at least one linking group, and/or at least one pendant fatty chain chosen from alkyl chains, for example alkyl chains comprising at least four carbon atoms, and alkenyl chains, for example, alkenyl chains comprising at least four carbon atoms, bonded to the at least one polyamide skeleton via at least one linking group. In one embodiment, the polyamide skeleton may comprise at least one terminal fatty chain chosen from fatty chains comprising 8 to 120 carbon atoms, such as, for example, 12 to 68 carbon atoms, bonded to the at least one polyamide skeleton via at least one linking group and/or at least one pendant fatty chain chosen from fatty chains comprising 8 to 120 carbon atoms, such as, for example, 12 to 68 carbon atoms, bonded to the at least one polyamide skeleton via at least one linking group, such as bonded to any carbon or nitrogen of the polyamide skeleton via said at least one linking group. In one embodiment, the at least one linking group is chosen from single bonds and urea, urethane, thiourea, thiourethane, thioether, thioester, ester, ether and amine groups. The bond is, for example, an ester bond. In one embodiment, these polymers comprise a fatty chain at each end of the polymer skeleton, such as the polyamide skeleton.

In one embodiment, due to the presence of at least one chain, the polyamide polymers may be readily soluble in oils (i.e., water-immiscible liquid compounds) and thus may give macroscopically homogeneous compositions even with a high content (at least 25%) of the polyamide polymers, unlike certain polymers of the prior art that do not contain such alkyl or alkenyl chains at the end of the polyamide skeleton. As defined herein, a composition is soluble if it has a solubility of greater than 0.01 g per 100 ml of solution at 25° C.

In a further embodiment, the polyamide polymers can be chosen from polymers resulting from at least one polycondensation reaction between at least one acid, chosen from dicarboxylic acids comprising at least 32 carbon atoms, such as 32 to 44 carbon atoms, and at least one amine, chosen from diamines comprising at least 2 carbon atoms, such as from 2 to 36 carbon atoms, and from triamines comprising at least 2 carbon atoms, such as from 2 to 36 carbon atoms. The at least one dicarboxylic acid can, for example, be chosen from dimers of at least one fatty acid comprising at least 16 carbon atoms, such as oleic acid, linoleic acid and linolenic acid. The at least one amine can, for example, be chosen from diamines, such as ethylenediamine, hexylenediamine, hexamethylenediamine, and phenylenediamine and from triamines, such as ethylenetriamine.

The polyamide polymers may also be chosen from polymers comprising at least one terminal carboxylic acid group. The at least one terminal carboxylic acid group can, for example, be esterified with at least one alcohol chosen from monoalcohols comprising at least 4 carbon atoms. For example, the at least one alcohol can be chosen from monoalcohols comprising from 10 to 36 carbon atoms. In a further embodiment, the monoalcohols can comprise from 12 to 24 carbon atoms, such as from 16 to 24 carbon atoms, and, for example, 18 carbon atoms.

In one embodiment, the at least one polyamide polymer may be chosen from those described in U.S. Pat. No. 5,783,657, the disclosure of which is incorporated herein by reference, which are polymers of formula (I):

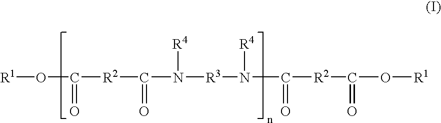

in which:

n is an integer which represents the number of amide units such that the number of ester groups present in said at least one polyamide polymer ranges from 10% to 50% of the total number of all said ester groups and all said amide groups comprised in the at least one polyamide polymer;

R¹, which are identical or different, are each chosen from alkyl groups comprising at least 4 carbon atoms and alkenyl groups comprising at least 4 carbon atoms. In one embodiment, the alkyl group comprises from 4 to 24 carbon atoms and the alkenyl group comprises from 4 to 24 carbon atoms;

R², which are identical or different, are each chosen from C₄ to C₄₂ hydrocarbon-based groups with the proviso that at least 50% of all R² are chosen from C₃₀ to C42 hydrocarbon-based groups;

R³ , which are identical or different, are each chosen from organic groups comprising atoms chosen from carbon atoms, hydrogen atoms, oxygen atoms and nitrogen atoms with the proviso that R³ comprises at least 2 carbon atoms; and

R⁴, which are identical or different, are each chosen from hydrogen atoms, C₁ to C₁₀ alkyl groups and a direct bond to at least one group chosen from R³ and another R⁴ such that when said at least one group is chosen from another R⁴, the nitrogen atom to which both R³ and R⁴ are bonded forms part of a heterocyclic structure defined in part by R⁴—N—R³, with the proviso that at least 50% of all R⁴ are chosen from hydrogen atoms.

In the polymer of formula (I), the terminal fatty chains that are optionally functionalized for the purposes of the invention are terminal chains linked to the last hetero atom, in this case nitrogen, of the polyamide skeleton.

In one embodiment, the ester groups of formula (I), which form part of the terminal and/or pendant fatty chains for the purposes of the invention, are present in an amount ranging from 15% to 40% of the total number of ester and amide groups (i.e. hetero atom groups), such as from 20% to 35%.

In formula (I), in one embodiment, n may be an integer ranging from 1 to 10, for example from 1 to 5, and as a further example an integer ranging from 3 to 5. In the present invention, R¹, which are identical or different, can, for example, each be chosen from C₁₂ to C₂₂ alkyl groups, such as from C₁₆ to C₂₂ alkyl groups.

In the present invention, R², which are identical or different, can, for example, each be chosen from C₁₀ to C₄₂ hydrocarbon-based, e.g., alkylene groups. At least 50% of all R², for example at least 75% of all R², which are identical or different, can, for example, each be chosen from groups comprising from 30 to 42 carbon atoms. In the two aforementioned embodiments, the remaining R², which are identical or different, can, for example, each be chosen from C₄ to C₁₈ groups, such as C₄ to C₁₂ groups.

R³, which can be identical or different, can, for example, each be chosen from C₂ to C₃₆ hydrocarbon-based groups and polyoxyalkylene groups. In another example, R³, which can be identical or different, can each, for example, be chosen from C₂ to C₁₂ hydrocarbon-based groups. In another embodiment, R⁴, which can be identical or different, can each be chosen from hydrogen atoms.

As used herein, hydrocarbon-based groups may be chosen from linear, cyclic and branched, and saturated and unsaturated groups. The hydrocarbon-based groups can be chosen from aliphatic and aromatic groups. In one example, the hydrocarbon-based groups are chosen from aliphatic groups. The alkyl and alkylene groups may be chosen from linear, cyclic and branched, and saturated and unsaturated groups.

In general, the pendant and terminal fatty chains may be chosen from linear, cyclic and branched, and saturated and unsaturated groups. The pendant and terminal fatty chains can be chosen from aliphatic and aromatic groups. In one example, the pendant and terminal fatty chains are chosen from aliphatic groups.

According to the invention, the structuring of the liquid fatty phase is obtained with the aid of at least one structuring polymer, such as the at least one polymer of formula (I). The at least one polyamide polymer of formula (I) may, for example, be in the form of a mixture of polymers, and this mixture may also comprise a compound of formula (I) wherein n is equal to zero, i.e., a diester.

Non-limiting examples of an at least one polyamide polymer which may be used in the composition according to the present invention include the commercial products sold or made by Arizona Chemical under the names Uniclear 80 and Uniclear 100. These are sold, respectively, in the form of an 80% (in terms of active material) gel in a mineral oil and a 100% (in terms of active material) gel. These polymers have a softening point ranging from 88° C. to 94° C., and may be mixtures of copolymers derived from monomers of (i) C₃₆ diacids and (ii) ethylenediamine, and have a weight-average molecular mass of about 6000. Terminal ester groups result from esterification of the remaining acid end groups with at least one alcohol chosen from cetyl alcohol and stearyl alcohol. A mixture of cetyl and stearyl alcohols is sometimes called cetylstearyl alcohol.

Other non-limiting examples of an at least one polyamide polymer which may be used in the composition according to the present invention include polyamide polymers (or polyamide resins) resulting from the condensation of at least one aliphatic dicarboxylic acid and at least one diamine, the carbonyl and amine groups being condensed via an amide bond. In one embodiment, these polymers contain more than two carbonyl groups and more than two amine groups. Examples of these polyamide polymers are those sold or made under the brand name Versamid by the companies General Mills Inc. and Henkel Corp. (Versamid 930, 744 or 1655) or by the company Olin Mathieson Chemical Corp. under the brand name Onamid, in particular Onamid S or C. These resins have a weight-average molecular mass ranging from 6000 to 9000. For further information regarding these polyamides, reference may be made to U.S. Pat. Nos. 3,645,705 and 3,148,125, the disclosures of which are hereby incorporated by reference. In one embodiment, Versamid 930 or 744 may be used.

Other examples of polyamides include those sold or made by the company Arizona Chemical under the references Uni-Rez (2658, 2931, 2970, 2621, 2613, 2624, 2665, 1554, 2623 and 2662) and the product sold or made under the reference Macromelt 6212 by the company Henkel. For further information regarding these polyamides, reference may be made to U.S. Pat. No. 5,500,209, the disclosure of which is hereby incorporated by reference. Such polyamides display high melt viscosity characteristics. MACROMELT 6212, for example, has a high melt viscosity at 190° C. of 30-40 poise (as measured by a Brookfield Viscometer, Model RVF #3 spindle, 20 RPM).

In a further embodiment, the at least one polyamide polymer may be chosen from polyamide resins from vegetable sources. Polyamide resins from vegetable sources may be chosen from, for example, the polyamide resins of U.S. Pat. Nos. 5,783,657 and 5,998,570, the disclosures of which are herein incorporated by reference.

The at least one structuring polymer in the composition of the invention may have a softening point greater than 50° C., such as from 65° C. to 190° C., and preferably less than 150° C., and further such as from 70° C. to 130° C., and even further such as from 80° C. to 105° C. This softening point may be lower than that of structuring polymers used in the art which may facilitate the use of the at least one structuring polymer of the present invention and may limit the degradation of the liquid fatty phase. These polymers may be non waxy polymers. The softening point can be measured by a well known method as “Differential Scanning Calorimetry (i.e. DSC method) with a temperature rise of 5 to 10° C./min.

In one embodiment, the at least one structuring polymer in the composition according to the invention corresponds to the polyamide polymers of formula (I). Due to fatty chain(s), these polymers may be readily soluble in oils and thus lead to compositions that are macroscopically homogeneous even with a high content (at least 25%) of at least one structuring polymer, unlike polymers not containing a fatty chain.

The at least one structuring polymer may be present in the composition in an amount ranging, for example, from 0.5% to 80% by weight relative to the total weight of the composition, such as, for example 2% to 60%, and further, for example, from 5 to 40%. In a further embodiment, the at least one structuring polymer may be present in the composition in an amount ranging, for example, from 5% to 25% by weight relative to the total weight of the composition.

In one embodiment, when the at least one structuring polymer of the present invention comprises a urea-urethane having the following formula:

R—O—CO—NH—R′—NH—CO—NH—R″—NH—CO—NH—R′—NH—CO—OR

then R represents CₙH₂ₙ₊₁, wherein n represents an integer having a value greater than 22, for example from 23 to 120, and further, for example from 23 to 68, or CₘH₂ₘ₊₁ (OCₚH₂ₚ)ᵣ—, wherein m represents an integer having a value of greater than 18, for example from 19 to 120, and further, for example, from 23 to 68, p represents an integer having a value of from 2 to 4, and r represents an integer having a value of from 1 to 10,

R′ represents:

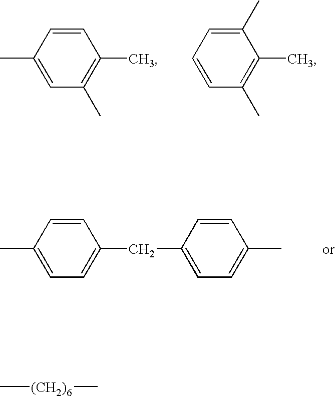

and R″ represents:

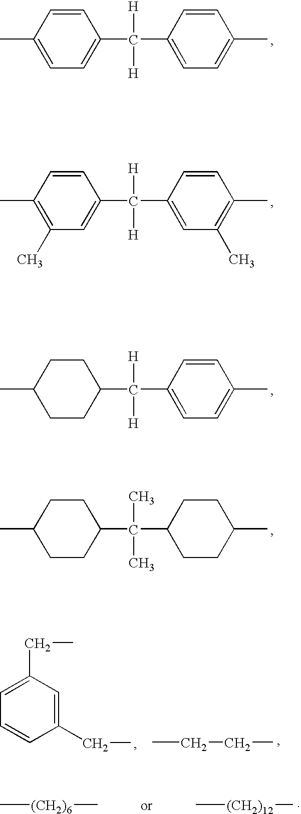

—CH₂—CH₂—, —(CH₂)₆— or —(CH₂)₁₂—.

In another embodiment of the invention, the present invention is drawn to a structured composition comprising at least one liquid fatty phase structured with at least one structuring polymer comprising a polymer skeleton comprising at least one hydrocarbon-based repeating unit comprising at least one hetero atom, wherein the at least one structuring polymer further comprises at least one terminal fatty chain, optionally functionalized, chosen from alkyl and alkenyl chains, such as alkyl and alkenyl chains having at least four carbon atoms, and further such as alkyl and alkenyl chains comprising, from 8 to 120 carbon atoms, bonded to the polymer skeleton via at least one linking group chosen from amides, ureas, and esters, wherein when said at least one linking group may be chosen from esters, said at least one terminal fatty chain is chosen from branched alkyl groups, and at least one organogelator. The at least one structuring polymer may also comprise at least one pendant fatty chain, optionally functionalized, chosen from alkyl and alkenyl chains, such as alkyl and alkenyl chains having at least four carbon atoms, and further such as alkyl and alkenyl chains comprising from 8 to 120 carbon atoms, bonded to any carbon or hetero atom of the polymer skeleton via at least one linking group chosen from amides, ureas, and esters, wherein when said at least one linking group is chosen from esters, said at least one terminal fatty chain may be chosen from branched alkyl groups. The at least one structuring polymer may comprise both at least one pendant fatty chain and at least one terminal fatty chain as defined above in this paragraph.

Further, an embodiment of the invention relates to a skin, lip, or keratinous fiber care, treatment, or make-up composition comprising a structured composition containing at least one liquid fatty phase structured with at least one structuring polymer comprising a polymer skeleton comprising at least one hydrocarbon-based repeating unit comprising at least one hetero atom and at least one organogelator able to gel the liquid fatty phase.

Additionally, an embodiment of the invention relates to a skin, lip, or keratinous fiber care or make-up composition comprising a structured composition containing at least one liquid fatty phase structured with at least one structuring polymer comprising a polymer skeleton comprising at least one hydrocarbon-based repeating unit comprising at least one hetero atom, at least one organogelator able to gel the liquid fatty phase, and at least one coloring agent.

Additionally, an embodiment of the invention relates to a method of making up skin or lips or making up keratinous fibers or caring for skin or lips or caring for keratinous fibers comprising applying to said skin, lips, or keratinous fibers a structured composition containing at least one liquid fatty phase structured with at least one structuring polymer comprising a polymer skeleton comprising at least one hydrocarbon-based repeating unit comprising at least one hetero atom and at least one organogelator able to gel the liquid fatty phase.

Another embodiment of the invention relates to a mascara, an eyeliner, a foundation, a lipstick, a blusher, a make-up-removing product, a make-up product for the body, a nail composition, an eyeshadow, a face powder, a concealer product, a shampoo, a conditioner, an antisun product or a care product for the lips, hair or nails comprising a composition comprising at least one liquid fatty phase in said mascara, eyeliner, foundation, lipstick, blusher, make-up-removing product, make-up product for the body, nail composition, eyeshadow, face powder, concealer product, shampoo, conditioner, antisun product or care product for the lips, hair or nails which comprises:

(i) at least one structuring polymer comprising:

a polymer skeleton which comprises at least one hydrocarbon-based repeating unit comprising at least one hetero atom; and

(ii) at least one organogelator.

Another embodiment of the invention relates to a deodorant product or a care product for the skin or body comprising an anhydrous composition comprising at least one liquid fatty phase in said product which comprises:

(i) at least one structuring polymer comprising:

a polymer skeleton which comprises at least one hydrocarbon-based repeating unit comprising at least one hetero atom; and

(ii) at least one organogelator.

In particular, the organogelator is not 12-hydroxystearic acid or methyl 12-hydroxystearate.

Another embodiment of the invention relates to a care and/or treatment and/or make-up composition for keratinous fibers, lips or skin comprising at least one liquid fatty phase in said care and/or treatment and/or make-up composition for keratinous fibers, lips or skin which comprises:

(i) at least one structuring polymer comprising:

a polymer skeleton which comprises at least one hydrocarbon-based repeating unit comprising at least one hetero atom; and

(ii) at least one organogelator.

Another embodiment of the invention relates to a lipstick composition in stick form comprising at least one continuous liquid fatty phase, at least one organogelator for the fatty phase and at least one non-waxy structuring polymer having a weight-average molecular mass of less than 1 00 000, said continuous liquid fatty phase, said at least one organogelator for the fatty phase and said at least one non-waxy structuring polymer being present in said lipstick composition.

Another embodiment of the invention relates to a method for care, make-up or treatment of keratin materials comprising applying to said keratin materials an anhydrous composition comprising at least one liquid fatty phase which comprises:

(i) at least one structuring polymer comprising:

a polymer skeleton which comprises at least one hydrocarbon-based repeating unit comprising at least one hetero atom; and

(ii) at least one organogelator.

In particular, the organogelator is not 12-hydroxystearic acid or methyl 12-hydroxystearate.

Another embodiment of the invention relates to a method for care, make-up or treatment of keratinous fibers, lips, or skin comprising applying to said keratinous fibers, lips, or skin a composition comprising at least one liquid fatty phase which comprises:

(i) at least one structuring polymer comprising:

a polymer skeleton which comprises at least one hydrocarbon-based repeating unit comprising at least one hetero atom; and

(ii) at least one organogelator.

Another embodiment of the invention relates to a method for providing an anhydrous composition having at least one property chosen from a solid appearance, non-exudation, shear-strength, gloss, and comfortable deposit on keratin materials chosen from lips, skin, and keratinous fibers, comprising including in said composition at least one liquid fatty phase which comprises:

(i) at least one structuring polymer comprising:

a polymer skeleton which comprises at least one hydrocarbon-based repeating unit comprising at least one hetero atom; and

(ii) at least one organogelator.

Another embodiment of the invention relates to a make up or care or treatment composition for the skin, the lips, or keratinous fibers comprising a structured composition containing at least one liquid fatty phase structured with at least one structuring polymer comprising a polymer skeleton comprising at least one hydrocarbon-based repeating unit comprising at least one hetero atom, at least one organogelator able to gel the liquid fatty phase, and at least one coloring agent.

Another embodiment of the invention relates to a method of making up or caring for skin, lips keratinous fibers comprising applying to said skin, lips, or keratinous fibers a structured composition containing at least one liquid fatty phase structured with at least one structuring polymer comprising a polymer skeleton comprising at least one hydrocarbon-based repeating unit comprising at least one hetero atom and at least one organogelator able to gel the liquid fatty phase.

Another embodiment of the invention relates to an anhydrous composition comprising at least one liquid fatty phase which comprises:

(i) at least one structuring polymer comprising:

a polymer skeleton which comprises at least three hydrocarbon-based repeating units comprising at least one hetero atom; and

(ii) at least one organogelator, and for example, the at least three hydrocarbon-based repeating units can be identical.

Another embodiment of the invention relates to a composition comprising at least one liquid fatty phase which comprises:

(i) at least one structuring polymer chosen from urea urethanes having the following formula XVI:

R—O—CO—NH—R′—NH—CO—NH—R″—NH—CO—NH—R′—NH—CO—OR (XVI)

wherein R represents CₙH₂ₙ₊₁—, wherein n represents an integer having a value greater than 22 or CₘH₂ₘ₊₁ (OCₚH₂ₚ)ᵣ—, wherein m represents an integer having a value of greater than 18, p represents an integer having a value of from 2 to 4, and r represents an integer having a value of from 1 to 10,

R′ represents:

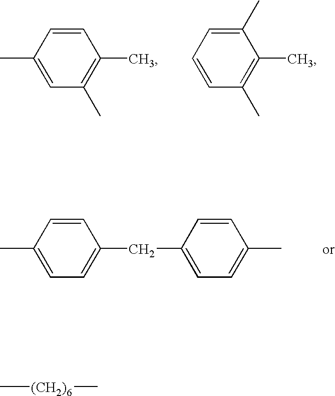

or —(CH₂)₆—

and R″ represents:

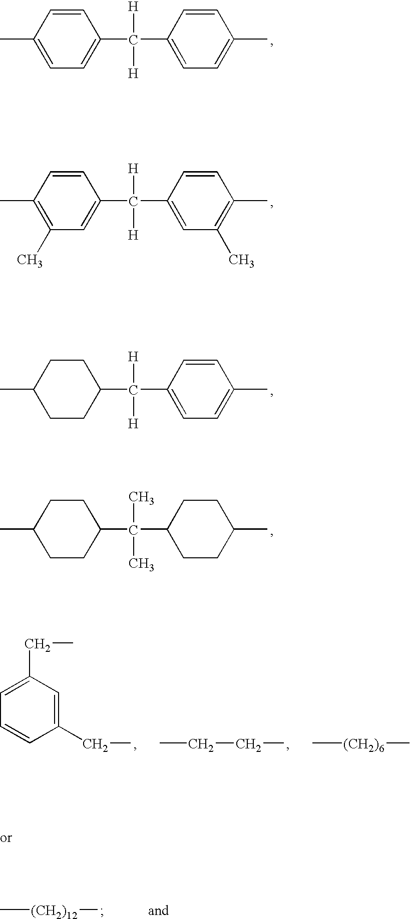

(ii) at least one organogelator.

Another embodiment of the invention relates to a composition comprising at least one liquid fatty phase which comprises:

(i) at least one structuring polymer comprising

a polymer skeleton which comprises at least one hydrocarbon-based repeating unit comprising at least one hetero atom with the proviso that said at least one hetero atom is not nitrogen; and

(ii) at least one organogelator.

Another embodiment of the invention relates to a composition comprising:

(i) at least one structuring polymer chosen from urea urethanes having the following formula XVII:

R—O—CO—NH—R′—NH—CO—NH—R″—NH—CO—NH—R′—NH—CO—OR (XVII)

wherein R represents CₙH₂ₙ₊₁—, wherein n represents an integer having a value of 4 from to 22 or CₘH₂ₘ₊₁ (OCₚH₂ₚ)ᵣ—, wherein m represents an integer having a value of from 1 to 18, p represents an integer having a value of from 2 to 4, and r represents an integer having a value of from 1 to 10.

R′ represents:

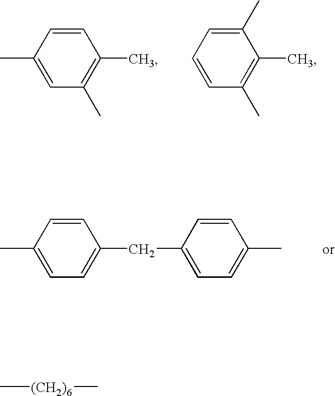

and R″ represents:

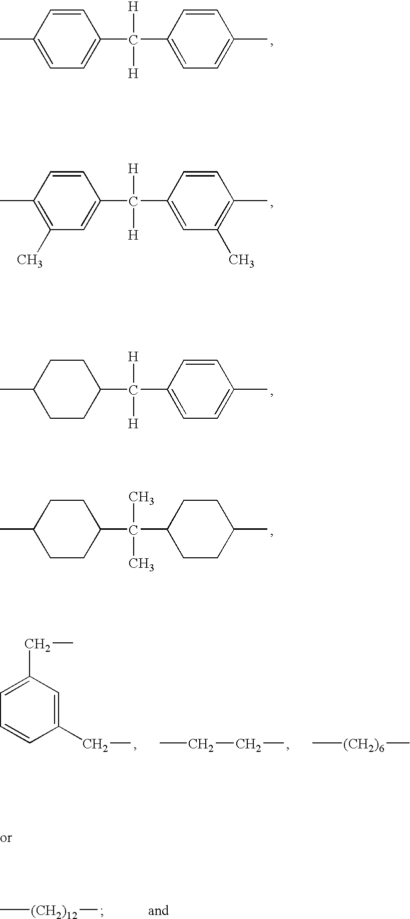

(ii) at least one organogelator.

Organogelator

According to the invention, the composition comprises at least one organogelator. An organogelator is defined herein to include a non-polymeric organic compound whose molecules may be capable of establishing, between themselves, at least one physical interaction leading to a self-aggregation of the molecules with formation of a macromolecular 3-D network which may be responsible for the gelation of the liquid fatty phase. The network can result from the formation of a network of fibrils (due to the stacking or aggregation of organic-gelling molecules), immobilizing the molecules of the liquid fatty phase. Depending on the nature of the organogelator, the interconnected fibrils have variable dimensions which may range up to one micron, or even several microns. These fibrils may occasionally combine to form strips or columns.

The term “gelation” means a thickening of the medium which may result in a gelatinous consistency and even in a solid, rigid consistency which does not flow under its own weight. The ability to form this network of fibrils, and thus the gelation, depends on the nature (or chemical category) of the organogelator, the nature of the substituents borne by its molecules for a given chemical category, and the nature of the liquid fatty phase. For example, this gelation is reversible.

The physical interactions are diverse but may exclude co-crystallization. These physical interactions are, for instance, interactions chosen from self-complementary hydrogen interactions, X interactions between unsaturated rings, dipolar interactions, and coordination bonding with organometallic derivatives. The establishment of these interactions may often be promoted by the architecture of the molecule, such as by rings, unsaturations, and the presence of asymmetric carbons. In general, each molecule of an organogelator can establish several types of physical interaction with a neighboring molecule. Thus, in one embodiment, the molecules of the organogelator according to the invention may comprise at least one group capable of establishing hydrogen bonding, e.g., at least two groups capable of forming hydrogen bonding; at least one aromatic ring, e.g., at least two aromatic rings; at least one bond with ethylenic unsaturation; and/or at least one asymmetric carbon. The groups capable of forming hydrogen bonding may, for example, be chosen from hydroxyl, carbonyl, amine, carboxylic acid, amide and benzyl groups.

The at least one organogelator of the invention may be soluble in the liquid fatty phase at room temperature and atmospheric pressure. They may be solid or liquid at room temperature and atmospheric pressure.

Organogelator(s) which can be used in the invention are, for example, those described in the document “Specialist Surfactants” edited by D. Robb, 1997, pp. 209-263, chapter 8, by P. Terech, and the French patent application nos. (FR-A-2796276) 99/09178 and 00/09317 (or FR-A-2811552), the disclosures of which are incorporated by reference herein. The organogelators described in these documents are, for example, chosen from:

hydroxylated carboxylic fatty acids having a linear or branched aliphatic carbon chain containing, in one embodiment, at least 8 carbon atoms, such as at least 12 carbon atoms, for instance 12-hydroxystearic acid and 12-hydroxyoleic acid and salts thereof, such as alkali metal salts (in particular Li, Na and K salts) and alkaline-earth metal (for example Mg) salts or esters thereof resulting from esterification of a mono alcohol or polyol having a linear or cyclic, saturated or not chain with from 1 to 6 carbon atoms;

amides of carboxylic acids, such as tricarboxylic acids, for instance the cyclohexanetricarboxamides (see patent application FR-A-2796276, the disclosure of which is incorporated by reference), these amides corresponding, for example, to formula (III) below;

amino acid amides or esters, for instance alanine esters and valine amides (such as those described in the book “Specialist Surfactants”);

N-acylamino acid amides, for instance the diamides resulting from the action of an N-acylamino acid with amines containing from 1 to 22 carbon atoms, such as those disclosed in document WO-93/23008, the disclosure of which is incorporated by reference, for example, N-acylglutamides in which the acyl group is a C₈ to C₂₂ alkyl chain, and N-laurylglutamic acid dibutylamide, such as the product sold or made by the company Ajinomoto under the name GP-1;

diamides having hydrocarbon-based chains each containing from 1 to 22 carbon atoms, for example, from 6 to 18 carbon atoms, these hydrocarbon-based chains being optionally substituted with ester, urea or fluoro groups (see patent application FR 00/09317, the disclosure of which is incorporated by reference), these diamides being, for example, those of formula (II) hereafter; and such as those resulting from the reaction of diaminocyclohexane, for example, trans-diaminocyclohexane, and of acid chloride;

steroid amines or amides, such as those from deoxycholic acid, cholic acid, apocholic acid or lithocholic acid and salts thereof, for instance D-17,17-dipropyl-17a-aza-5α-homoandrostan-3β-ol or D-17,17-dipropyl-17a-aza-5α-homoandrostan-3β-ol 17a-oxy;

compounds containing several aromatic rings (2 or 3), such as anthryl derivatives comprising at least 2 alkyl chains containing from 8 to 30 carbon atoms, for instance 2,3-bis(n-decyloxy)anthracene or 2,3-bis(n-decyloxy)anthraquinone, or comprising a steroid group, for instance cholesteryl 4-(2-anthryloxy)butanoate or cholesteryl anthraquinone-2-carboxylate and derivatives thereof;

azobenzene steroids such as those described in the book “Specialist Surfactants”;

organometallic compounds, for instance mononuclear copper β-diketonate (the octasubstituted copper complex of bis(3,4-nonyloxybenzoyl) methanes), binuclear copper tetracarboxylates or the Zn (II) complexes of trisubstituted (para-carboxyphenyl)porphyrine;

surfactants in salt form comprising at least two linear or branched alkyl chains, such as alkali metal or aluminium alkyl phosphates comprising two alkyl chains containing from 8 to 30 carbon atoms, for instance the aluminium salt of hexadecyl phosphate (C₁₆DP-Al) or bis(2-ethylhexyl)phosphate and alkali metal (Na) salts thereof, bis(2-ethylhexyl) sulphosuccinate and the alkali metal (Na) salts thereof;

benzylidene sorbitols or alditols and derivatives thereof, for instance 1,3:2,4-di-o-benzylidene-D-sorbitol;

cyclodipeptides which are cyclic condensates of two amino acids such as those disclosed in the book “Specialist Surfaotants”;

cyclic compounds or alkylene compounds comprising two urea or urethane groups such as dialkylurea cyclohexane, having, for example, the formula (IV) below;

alkylaryl cyclohexanol derivatives in which the alkyl chain is linear or branched and comprises from 1 to 22 carbon atoms and the aryl portion is, for example, a phenyl group, these derivatives being, for instance, 4-tert-butyl-1-phenyl cyclohexanol;

callixarenes such as those mentioned in the book “Specialist Surfactants”;

associations of 2,4,6-tri-aminopyrimidine substituted by an alkyl chain and dialkyl barbituric acid, the alkyl chains of which are linear or branched and comprise from 1 to 22 carbon atoms;

compounds such as those described in the document WO-A-01/07007, the disclosure of which is herein incorporated by reference, and having the following formula (V)

Q-O—W—(CHOH)ₛ—W¹—O-Q¹ (V)

in which W and W¹, which may be identical or different, are chosen from —CH₂—, —CO— and in which Q and Q¹, which may be identical or different, are a hydrocarbon-based chain chosen from saturated or unsaturated linear or branched hydrocarbon-based chains containing at least 6 carbon atoms, and in which s is an integer from 2 to 4; such as the compounds in which W═W¹═—CH₂— and s=2 and the compounds in which W═W¹═—CO— and s=4;

gluconamide derivatives such as those disclosed in the article R. J. H HAFKAMP, Chem. Commun., (1997), pages 545-46 and in the article, J. org. Chem, vol 64, N°2; 412-26 (1999), the disclosures of which are herein incorporated by reference and having a formula (VI):

R₁—NH—CO—[CH(OH)]₄—CH₂R₂ (VI)

in which R¹ is a hydrocarbon-based chain chosen from saturated or unsaturated linear, branched and cyclic hydrocarbon-based chains having 1 to 30 carbon atoms ; this hydrocarbon-based chain optionally can comprise at least one hetero atom such as N, O and S; and for example the compounds in which R₂═—O—CO—R₃ or —O—R₃ with R₃ chosen from linear and branched alkyl chains containing 1 to 20 carbon atoms, C₅-C₈ cycloaliphatic and aromatic chains, C₅-C₈ heterocycles comprising N, O or S atoms, and for example the compounds in which R₂ is a C₅-C₈ saturated or unsaturated heterocycles comprising N, O, S atom such as R₂ is imidazolyl group; and

cyclic ether derivatives of compound of formula VI, having the formula VI′:

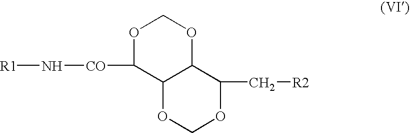

wherein R₁ and R₂ has the same meaning as defined in formula (VI).

bis oxalylamides of aminoacides such as those mentioned in the article M. JOKIC, J. chem. soc., chem. commun., pages 1723-24 (1995), the disclosure of which is herein incorporated by reference, and for example having the formula VII:

HOCO—CH(R₁)—NH—CO—CO—NH—CH(R₂)—COOH (VII)

in which R₁ and R₂ may be identical or different, are a group chosen from

—CH₂—CH(CH₃)₂; —C₆H₅; —CH₂—C₆H₅; —CH(CH₃)₂;

amide and urea derivatives of lysine ester such as those mentioned in the article K. HANABUSA, Chemistry Letters, p1070-71 (2000), the disclosure of which is herein incorporated by reference, such as N⁶⁸ _lauroyl-N^{α}-stearyl aminocarbonyl-L-lysine (ethyl or methyl) ester and derivatives having a formula:

C₁₁—H₂₃—CO—NH—(CH₂)₄—CH(COOR₁)—NH—CO—R₂;

in which R₁═—CH₃ or —C₂H₅ and R₂═—NH—(CH₂)₁₇—CH₃, —NH—(CH₂)ₙ—CH₃

derivatives from diamides benzene dicarboxylic of acides and valine such as those mentioned in the article K. HANABUSA, Chemistry Letters, 767-8 (1999), the disclosure of which is herein incorporated by reference, and for example:

in which -L-Val-represents: —NH—CH(CH(CH₃)₂)—CO—;

monoalkyloxamides such as those disclosed by X. LUO, Chem. Commun., 2091-92, (2000), the disclosure of which is herein incorporated by reference, and for example having the formula:

R₁—NH—CO—CO—NH—R₂

In which R₁ and R₂ which can be identical or different are a hydrocarbon-based chain chosen from saturated or unsaturated linear, branched and cyclic hydrocarbon-based chains having 1 to 30 carbon atoms;

bolaamphiphiles having 1 -glucosamide head, such as N,N′-bis(β-D-glucopyranosyl)alcane-1, n-dicarboxamide, these compounds being mentioned in the article T. SHIMIZU, J. Am. Chem. Soc., 119, 2812-18 (1997), the disclosure of which is herein incorporated by reference, and has the formula VIII:

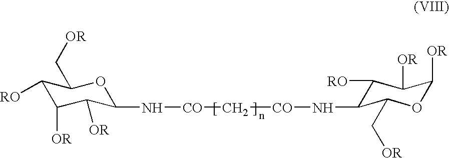

in which n is an integer from 2 to 30, R is —H or —CO—R₁ in which R₁ is a C₁-C₂₀ alkyl group, and for example the compound in which R═—CO—CH₃

alkyl-2-ammonium-2-isobutylacétate p-toluène sulfonate such as those disclosed by K. HANABUSA, Collöid Polym. Sci, 276, 252-59 (1998), the disclosure of which is herein incorporated by reference, and having the formula XII:

p-CH₃—C₆H₄—SO₃⁻ ⁺H₃N—CH(R₁)—CO—OR₂ (XII)

in which R₁═—CH₂—CH(CH₃)₂; —CH(CH₃)₂; —CH(CH₃)—CH₂—CH₃; —CH₂—C₆H₅—CH₂—CH₂—CO—O—CH₂—(CH₂)₁₀—CH₃

and R₂═—CH₂—(CH₂)ₙ—CH₃ with n an integer from 4 to 12.

—(CH₂)₂—CH(CH₃)—(CH₂)₃—CH(CH₃)₂

cellobiose fatty esters, such a those mentioned in WO-A-00/61080,

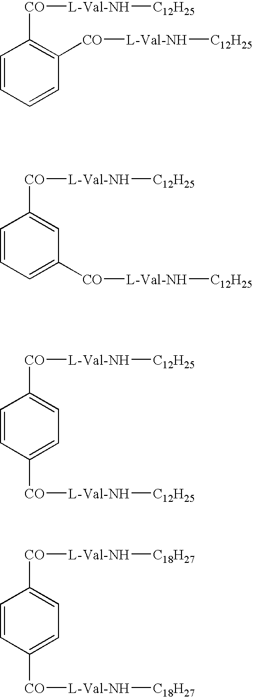

the disclosure of which is herein incorporated by reference, and WO-A-00/61081, the disclosure of which is herein incorporated by reference, and having the formula XIII:

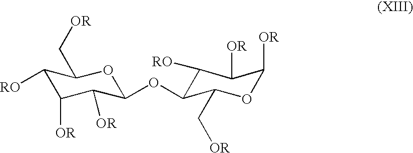

in which R═—CO—R1 and R1=alkyl or alkylene group with 5 to 12 carbon atoms.

diamides having the formula XIV or XV

R₂—X—CO—NH—R₁—NH—CO—X—R₂ (XIV) or

R₂—CO—NH—R₁—NH—CO—R₂ (XV)

in which R₁ is alkylene group chosen from C₁-C₅₀ linear, branched and cyclic groups and C₅-C₈ arylene groups and alkylen groups comprising C₁-C₄ alkyl group; and in which —X— represents —O— or —NH—; and in which R₂, which may be identical or different is a C₈-C₆₀ saturated or unsaturated linear or branched hydrocarbon-based chain, at least one R₂ comprising optionally a hydroxyl group or at least one hetero atom such as N, O, S or Si.

and mixtures thereof.

In one embodiment, amino acid amides such as N-acylamino acids and cyclohexane tricarboxamides, and mixtures thereof, are used.

Organogelator of Formula (II)

According to the invention, the organogelator may be a compound of formula (II) below:

R—CO—NH-A-NH—CO—R′

in which:

R and R′, which may be identical or different, are chosen from a hydrogen atom and hydrocarbon-based chains chosen from saturated linear, saturated branched, saturated cyclic, unsaturated linear, unsaturated branched and unsaturated cyclic hydrocarbon-based chains containing from 1 to 22 carbon atoms, for example from 6 to 18 carbon atoms, such as from 10 to 14 carbon atoms, optionally substituted with at least one group chosen from aryl (—C₆H₅), ester (—COOR″ with R″ being an alkyl group containing 2 to 12 carbon atoms), amide (—CONHR″ with R″ being an alkyl group containing from 2 to 12 carbon atoms), urethane (—OCONHR″ with R″ being an alkyl group containing from 2 to 12 carbon atoms) and urea (—NHCONHR″ with R″ being an alkyl group containing from 2 to 12 carbon atoms) groups; and/or optionally containing from 1 to 3 hetero atoms chosen from O, S and N; and/or optionally substituted with from 1 to 4 halogen atoms, in particular fluorine atoms, and/or with from 1 to 3 hydroxyl radicals,

with the proviso that R and/or R′ is other than hydrogen, and

A is chosen from saturated and unsaturated, linear, cyclic and branched hydrocarbon-based chains containing from 1 to 18 carbon atoms, such as from 2 to 12 carbon atoms, and for example from 4 to 12 carbon atoms, optionally substituted with at least one group chosen from aryl (—C₆H₅), ester (—COOR″ with R″ being an alkyl group containing from 2 to 12 carbon atoms), amide (—CONHR″ with R″ being an alkyl group containing from 2 to 12 carbon atoms), urethane (—OCONHR″ with R″ being an alkyl group containing from 2 to 12 carbon atoms) and urea (—NHCONHR″ with R″ being an alkyl group containing from 2 to 12 carbon atoms) groups; and/or optionally containing from 1 to 3 hetero atoms chosen from O, S and N; and/or optionally substituted with from 1 to 4 halogen atoms, such as fluorine atoms, and/or with from 1 to 3 hydroxyl radicals.

According to formula (II), the expression “unsaturated hydrocarbon-based chain” means a chain which comprises at least one C═C double bond or at least one C≡C triple bond, it being possible for the chain also to be optionally substituted with at least one group chosen from aryl, ester, amide, urethane and urea groups; and/or optionally to comprise at least one hetero atom chosen from O, S and N; and/or optionally to be substituted with at least one fluorine atom and/or hydroxyl radical. The expression “hydrocarbon-based chain according to formula (II) comprising an oxygen, sulphur or nitrogen atom” includes, in particular, a hydrocarbon-based chain comprising a carbonyl (C═O), amine (—NH₂ or —NH—), thiol (—SH), thioether or ether group.

The compounds, for example, correspond to the formula (II) in which:

1/

A is chosen from saturated and unsaturated but non-aromatic, optionally branched hydrocarbon-based rings containing from 4 to 12 carbon atoms, for example from 5 to 7 carbon atoms, optionally substituted with the substituents mentioned above and/or optionally comprising at least one hetero atom and/or optionally substituted with at least one halogen and/or hydroxyl radical;

R and R′, which may be identical or different, are chosen from a hydrogen atom and hydrocarbon-based chains chosen from saturated linear, saturated branched, saturated cyclic, unsaturated linear, unsaturated branched and unsaturated cyclic hydrocarbon-based chains containing from 10 to 16 carbon atoms, for example, from 12 to 14 carbon atoms, such as a saturated, linear hydrocarbon-based chain; or

2/

A is a saturated hydrocarbon-based chain chosen from linear and branched saturated hydrocarbon-based chains containing from 2 to 18 carbon atoms, for example from 3 to 12 carbon atoms, optionally substituted with the substitutents mentioned above, and/or optionally comprising at least one hetero atom and/or optionally substituted with at least one halogen and/or hydroxyl radical;

R and R′, which may be identical or different, are chosen from a hydrogen atom and a hydrocarbon-based chain chosen from saturated linear, saturated branched, saturated cyclic, unsaturated linear, unsaturated branched and unsaturated cyclic hydrocarbon-based chains, such as saturated, linear, hydrocarbon-based chains containing from 10 to 20 carbon atoms, for example, from 11 to 18 carbon atoms;

or alternatively

3/

A is chosen from aryl and aralkyl rings containing from 4 to 12 carbon atoms, for instance from 5 to 8 carbon atoms, optionally substituted with the substituents mentioned above and/or optionally comprising at least one hetero atom and/or optionally substituted with at least one halogen and/or hydroxyl radical;

R and R′, which may be identical or different, are chosen from a hydrogen atom and hydrocarbon-based chains chosen from saturated linear, saturated branched, saturated cyclic, unsaturated linear, unsaturated branched and unsaturated cyclic hydrocarbon-based chains, such as a saturated, linear, hydrocarbon-based chain, containing from 6 to 18 carbon atoms, for example from 10 to 16 carbon atoms.

The radical A may be, for example, a divalent radical such as cyclohexylene, ethylene, propylene, isopropylene, butylene, isobutylene, pentylene, hexylene, dodecylene, dodecanylene, benzylene, phenylene, methylphenylene, bis-phenylene or naphthalene.

The radicals R and R′ may be chosen, independently of each other, from, for example, pentyl, hexyl, decyl, undecyl, dodecyl, pentadecyl, hexadecyl, heptadecyl, octadecyl, 3-dodecyloxypropionyl, 3-octadecyloxy-propionyl, 3-dodecyloxypentyl, 3-octadecyloxypentyl and 11-hydroxyheptadecyl radicals. In one embodiment R and R′ are identical.

When the radical A is cyclic, the radicals R—CO—NH— and R′—CO—NH— may be in an ortho, meta or para position. Moreover, they may be in a cis or trans position relative to each other. In one embodiment, the compounds of formula (II) is a mixture of cis and trans compounds.

The compounds of formula (II) may be chosen from the compounds corresponding to one of the following formulae:

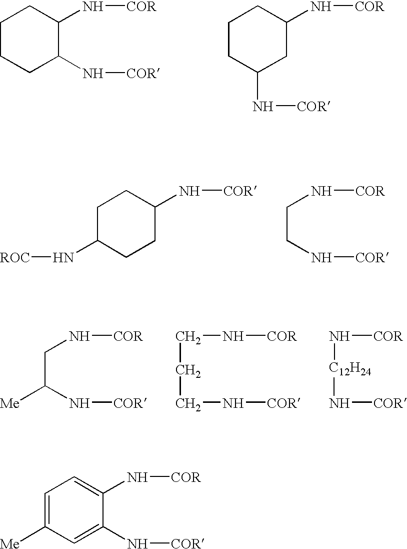

in which R and R′ have the same meanings as above.

Among the compounds which may be used as organogelators in the composition of the invention, mention may be made of:

N,N′-bis(dodecanoyl)-1,2-diaminocyclohexane, in particular in trans form (compound of formula (II) with R═R′=n-C₁₁H₂₃ and A=1,2-cyclohexylene divalent radical, also known as (2-dodecanoylaminocyclohexyl) dodecanamide. This compound is described in particular in Hanabusa, K; Angew. Chem., 108, 1997, 17, pages 2086-2088,

N,N′-bis(dodecanoyl)-1,3-diaminocyclohexane, in particular in trans form (compound of formula (II) with R═R′=n-C₁₁H₂₃ and A=1,3-cyclohexylene divalent radical, also known as (3-dodecanoylaminocyclohexyl-dodecanamide),

N,N′-bis(dodecanoyl)-1,4-diaminocyclohexane, in particular in trans form (compound of formula (II) with R=n-C₁₁H₂₃ and A=1,4-cyclohexylene divalent radical, also known as (4-dodecanoylaminocyclohexyl)dodecanamide),

N,N′-bis(dodecanoyl)-1,2-ethylenediamine (compound of formula (II) with R═R′=n-C₁₁H₂₃ and A=1,2-ethylene divalent radical, also known as (2-dodecanoylaminoethyl)dodecanamide),

N,N′-bis(dodecanoyl)-1-methyl-1,2-ethylenediamine (compound of formula (II) with R═R′=n-C₁₁H₂₃ and A=1-methyl-1,2-ethylene divalent radical, also known as (2-dodecanoylamino-2-methylethyl) dodecanamide),

N,N′-bis(dodecanoyl)-1,3-diaminopropane (compound of formula (II) with R═R′=n-C₁₁H₂₃ and A=1,3-propylene divalent radical, also known as (2-dodecanoylaminopropyl) dodecanamide),

N,N′-bis(dodecanoyl)-1,12-diaminododecane (compound of formula (II) with R═R′=n-C₁₁H₂₃ and A=1,12-dodecylene divalent radical, also known as (2-dodecanoylaminododecyl)dodecanamide),

N,N′-bis(dodecanoyl)-3,4-diaminotoluene (compound of formula (II) with R═R′=n-C₁₁H₂₃ and A=1-methyl-3,4-phenylene divalent radical, also known as (2-dodecanoylamino-4-methylphenyl)dodecanamide).

The compounds of formula (II) can be prepared according to processes that are well known to those skilled in the art.

In particular, they may be obtained by reacting a diamine H₂N-A-NH₂ with an acid chloride RCOCl and/or R′COCl with R and R′ having the above meaning, but other than a hydrogen atom, in an organic solvent medium which is compatible for carrying out the reaction (1 mol of acid chloride is used per 1 mol of diamine if it is desired to obtain a compound of formula (I) containing only one group R other than a hydrogen atom, or 2 mol of acid chloride RCOCl and/or R′COCl if it is desired to obtain a compound of formula (II) with R and R′ other than a hydrogen atom). The reaction is preferably carried out in the presence of a base capable of neutralizing the formation of the HCl released during the reaction. The diamide formed is extracted from the reaction medium according to the conventional extraction techniques that are well known to those skilled in the art.

The compounds of formula (II) can be prepared according to processes that are well known to those skilled in the art and can be used, alone or as a mixture, in the composition of the invention.

Standard Preparation of the Compounds of Formula (II) for R═R′

The diamine and two equivalents of triethylamine are dissolved in 50 ml of tetrahydrofuran. Two equivalents of acyl chloride dissolved in THF are added and the reaction mixture is heated to the reflux point of the tetrahydrofuran, while monitoring the disappearance of the acyl chloride by infrared spectroscopy (most typically, two hours). The solution is filtered from the precipitate, the organic phase is concentrated and a liquid/liquid extraction is performed on the solid compound obtained. The organic phase is subsequently dried and then concentrated, and the solid product obtained is recrystallized.

Organogelator of Formula (II)

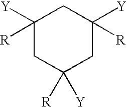

in which:

R is identical or different and each is chosen from a hydrogen atom, a saturated linear hydrocarbon-based chain, and a saturated branched hydrocarbon-based chain, wherein said hydrocarbon-based chains contain from 1 to 6 carbon atoms, for example from 1 to 4 carbon atoms;

Y is identical or different and each is a group chosen from the following groups: —CO—S—R′; —CO—NHR′; —NH—COR′ and —S—COR′; in which R′ is identical or different and each is chosen from:

a hydrogen atom;

an aryl group;

an aralkyl group, i.e., an aryl group substituted with a hydrocarbon-based chain chosen from saturated, linear hydrocarbon-based chains and saturated, branched hydrocarbon-based chains, wherein the hydrocarbon based chain contains from 1 to 22 carbon atoms, for example from 10 to 18 carbon atoms; and

a saturated hydrocarbon-based chain chosen from linear, branched and cyclic hydrocarbon-based chains containing from 1 to 22 carbon atoms, for example from 10 to 18 carbon atoms, optionally substituted with at least one group chosen from aryl, ester, amide and urethane groups; and/or optionally comprising at least one hetero atom chosen from O, S and N; and/or optionally substituted with at least one fluorine atom and/or hydroxyl radical.

R, for example, is chosen from a hydrogen atom.

Y, for example, is chosen from the groups —CO—NHR′ and —NH—COR′.

R′, for example, is chosen from an aryl group; an aralkyl group in which the linear or branched alkyl chain contains from 12-16 carbon atoms; and a linear or branched C₁₁-C₁₈ alkyl chain.

In one embodiment, Y is chosen from a group —CO—NHR′ in which R′ is chosen from an aryl group substituted with a C₁₂-C₁₆ alkyl chain chosen from linear and branched C₁₁-C₁₆ alkyl chains; or R′ is chosen from an unsubstituted linear C₁₁-C₁₈ alkyl chain and an unsubstituted branched C₁₁-C₁₈ alkyl chain.

The three substitutents represented by Y can be, in the compounds of formula (III), in cis-cis, cis-trans or trans-trans conformation relative to each other. In particular, at least one of these substituents may be placed in an equatorial position on the cyclohexane ring; for example, all the substituents Y are placed in an equatorial position. In one embodiment, the compounds of formula (III) is a mixture of cis-cis, cis-trans and/or trans-trans compounds.

Among the compounds of formula (Ill) which can be used as an organogelator, alone or as a mixture, in the composition of the invention, mention may be made of:

cis-1,3,5-tris(dodecylaminocarbonyl)cyclohexane,

cis-1,3,5-tris(octadecylaminocarbonyl)cyclohexane,

cis-1,3,5-tris[N-(3,7-dimethyloctyl)-aminocarbonyl]cyclohexane,

trans-1,3,5-trimethyl-1,3,5-tris(dodecylaminocarbonyl)cyclohexane, and

trans-1,3,5-trimethyl-1,3,5-tris(octadecylaminocarbonyl)cyclohexane.

The compounds of formula (III) are well known to those skilled in the art and can be prepared according to the usual processes.

It is also possible to add to the composition an organic compound as set forth in U.S. Pat. No. 6,156,325, the disclosure of which is incorporated by reference herein. Such compounds include urea urethanes having the following formula:

R—O—CO—NH—R′—NH—CO—NH—R″—NH—CO—NH—R′—NH—CO—OR

wherein R represents CₙH₂ₙ₊₁— or CₘH₂ₘ₊₁ (CₚH₂ₚO)ᵣ—; n represents an integer having a value of from 4 to 22; m represents an integer having a value of from 1 to 18; p represents an integer having a value of from 2 to 4; and r represents an integer having a value of from 1 to 10,

R′ represents:

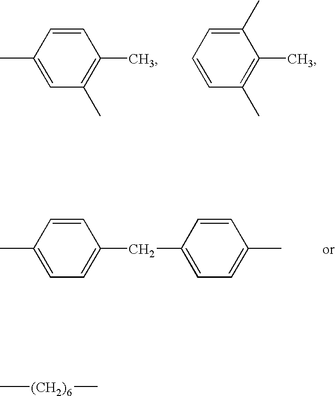

and R″ represents:

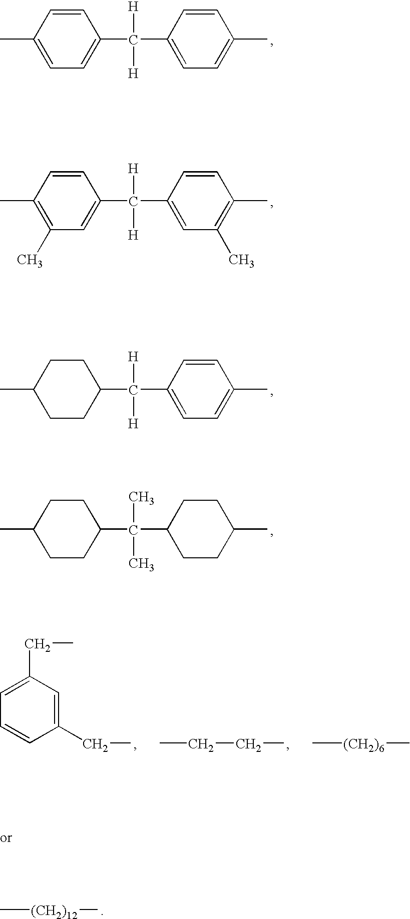

As is evident from the urea urethane formula above, the alkyl groups and alkyl portions designated for the R variable are saturated.

Organogelator of Formula (IV)

According to the invention the organogelator may be at least one organogelator of formula (IV);

RNHCONHANHCONHR

wherein A and R have the same definition as the one provided above for formula (11), expressed most broadly as:

R, which may be identical or different, is each chosen from a hydrogen atom and hydrocarbon-based chains chosen from saturated linear, saturated branched, saturated cyclic, unsaturated linear, unsaturated branched and unsaturated cyclic hydrocarbon-based chains containing from 1 to 22 carbon atoms, for example from 6 to 18 carbon atoms, optionally substituted with at least one group chosen from aryl (—C₆H₅), ester (—COOR″ with R″ being an alkyl group containing from 2 to 12 carbon atoms), amide (—CONHR″ with R″ being an alkyl group containing from 2 to 12 carbon atoms), urethane (—OCONHR″ with R″ being an alkyl group containing from 2 to 12 carbon atoms) and urea (—NHCONHR″ with R″ being an alkyl group containing from 2 to 12 carbon atoms) groups; and/or optionally containing from 1 to 3 hetero atoms chosen from O, S and N; and/or optionally substituted with from 1 to 4 halogen atoms, in particular fluorine atoms, and/or with from 1 to 3 hydroxyl radicals,

with the proviso that at least one R is other than hydrogen, and

A is chosen from saturated and unsaturated, linear, cyclic and branched hydrocarbon-based chains containing from 1 to 18 carbon atoms, such as from 2 to 12 carbon atoms, optionally substituted with at least one group chosen from aryl (—C₆H₅), ester (—COOR″ with R″ being an alkyl group containing from 2 to 12 carbon atoms), amide (—CONHR″ with R″ being an alkyl group containing from 2 to 12 carbon atoms), urethane (—OCONHR″ with R″ being an alkyl group containing from 2 to 12 carbon atoms) and urea (—NHCONHR″ with R″ being an alkyl group containing from 2 to 12 carbon atoms) groups; and/or optionally containing from 1 to 3 hetero atoms chosen from O, S and N; and/or optionally substituted with from 1 to 4 halogen atoms, such as fluorine atoms, and/or with from 1 to 3 hydroxyl radicals.

In one embodiment, the inventive composition contains from 0.1% to 80% by weight of organogelator. In another embodiment, the composition contains from 0.5% to 60% by weight of organogelator, for example, from 1% to 40% or from 2% to 30%.

Amphiphilic Compound

The at least one structuring polymer and the at least one organogelator can be combined with at least one amphiphilic compound that is liquid and non-volatile at room temperature and has a hydrophilic/lipophilic balance (HLB) value of less than 12, for example ranging from 1 to 8 or from 1 to 5. These amphiphilic compounds may act to reinforce the structuring properties of the polymer containing at least one hetero atom, to facilitate the implementation of the polymer and to improve the ability of the stick to be deposited. However, it is possible to obtain a stick with good mechanical and/or thermal properties without including at least one amphiphilic compound.

Depending on the intended application, such as a stick, hardness of the composition may also be considered. The hardness of a composition may, for example, be expressed in grams force (gf). The composition of the present invention may, for example, have a hardness ranging from 20 gf to 2000 gf, (196 to 19 600 N) such as from 20 gf to 900 gf (196 to 8 820 N), and further such as from 20 gf to 600 gf (196 to 5880 N).

This hardness is measured in one of two ways. A first test for hardness is according to a method of penetrating a probe into said composition and in particular using a texture analyzer (for example TA-XT2i from Rheo) equipped with an ebonite cylinder of height 25 mm and diameter 8 mm. The hardness measurement is carried out at 20° C at the center of 5 samples of said composition. The cylinder is introduced into each sample of composition at a pre-speed of 2 mm/s and then at a speed of 0.5 mm/s and finally at a post-speed of 2 mm/s, the total displacement being 1 mm. The recorded hardness value is that of the maximum peak observed. The measurement error is ±50 gf.

The second test for hardness is the “cheese wire” method, which involves cutting an 8.1 mm or preferably 12.7 mm in diameter stick of composition and measuring its hardness at 20° C. using a DFGHS 2 tensile testing machine from Indelco-Chatillon Co. at a speed of 100 mm/minute. The hardness value from this method is expressed in grams as the shear force required to cut a stick under the above conditions. According to this method, the hardness of compositions according to the present invention which may be in stick form may, for example, range from 30 gf to 300 gf (294 N to 2 940 N), such as from 30 gf to 250 gf (294 N to 2 450 N), and further such as from 30 gf to 200 gf (294 N to 1 960 N).

The hardness of the composition of the present invention may be such that the compositions are self-supporting and can easily disintegrate to form a satisfactory deposit on a keratinous material. In addition, this hardness may impart good impact strength to the inventive compositions which may be molded or cast, for example, in stick or dish form.

The skilled artisan may choose to evaluate a composition using at least one of the tests for hardness outlined above based on the application envisaged and the hardness desired. If one obtains an acceptable hardness value, in view of the intended application, from at least one of these hardness tests, the composition falls within the scope of the invention.

As is evident, the hardness of the composition according to the invention may, for example, be such that the composition is advantageously self-supporting and can disintegrate easily to form a satisfactory deposit on the skin and/or the lips and/or superficial body growths, such as keratinous fibres. In addition, with this hardness, the composition of the invention may have good impact strength.

According to the invention, the composition in stick form may have the behavior of a deformable, flexible elastic solid, giving noteworthy elastic softness on application. The compositions in stick form of the prior art do not have these properties of elasticity and flexibility.

The at least one amphiphilic compound which can be used in the composition of the invention may, for example, comprise a lipophilic part linked to a polar part, the lipophilic part comprising a carbon-based chain containing at least 8 carbon atoms, for example from 18 to 32 carbon atoms or from 18 to 28 carbon atoms. The polar part of the at least one amphiphilic compound may, in one embodiment, be the residue of a compound chosen from alcohols and polyols containing from 1 to 12 hydroxyl groups, and polyoxyalkylenes comprising at least 2 oxyalkylene units and containing from 0 to 20 oxypropylene units and/or from 0 to 20 oxyethylene units. For example, the at least one amphiphilic compound may be an ester chosen from the hydroxystearates, oleates and isostearates of glycerol, of sorbitan and of methylglucose, and from branched C₁₂ to C₂₆ fatty alcohols such as octyldodecanol. Among these esters, monoesters and mixtures of mono- and diesters can also be used.

The respective contents of the at least one lipophilic organogelator, the at least one polymer containing a hetero atom and optionally that of at least one amphiphilic compound are chosen according to the desired hardness of the composition and as a function of the specific application envisaged. The respective amounts of polymer, of organogelator and of amphiphilic compound should be such that they produce a stick which can be worn down. In practice, the amount of the at least one polymer may be chosen from 0.5% to 80% of the total weight of the composition, for example from 2% to 60%, from 5% to 40%, and from 5% to 25%. The amount of at least one amphiphilic compound in practice, if it is present, may be chosen from 0.1% to 35% of the total weight of the composition, for example from 1% to 20% or from 1% to 15%.

The at least one organogelator and/or the at least one structuring polymer have an affinity with the fatty phase and in particular with a chemical portion of one of the oils forming the liquid fatty phase of the composition so that physical links with the oils, such as hydrogen bonds or as above-mentioned, are formed.

Liquid Fatty Phase

The at least one liquid fatty phase, in one embodiment, may comprise at least one oil. In one embodiment, at least one oil has an affinity with the structuring polymer and/or with the organogelator. The at least one oil, for example, may be chosen from polar oils and apolar oils including hydrocarbon-based liquid oils and oily liquids at room temperature. In one embodiment, the composition of the invention comprise at least one structuring polymer and at least one polar oil. The polar oils of the invention, for example, may be added to the apolar oils, the apolar oils acting in particular as co-solvent for the polar oils.

According to the invention, the structuring of the at least one liquid fatty phase may be obtained with the aid of at least one structuring polymer, such as the polymer of formula (I). In general, the polymers of formula (I) may be in the form of mixtures of polymers, these mixtures also possibly containing a synthetic product corresponding to a compound of formula (I) in which n is 0, i.e., a diester.

The liquid fatty phase of the composition may contain more than 30%, for example, more than 40%, of liquid oil(s) having a chemical nature close to the chemical nature of the skeleton (hydrocarbon or silicone based) of the structuring polymer, and for example from 50% to 100%. In one embodiment, the liquid fatty phase structured with a polyamide-type skeleton or polyurea or polyurethane or polyurea-urethane-type skeleton contains a high quantity, i.e., greater than 30%, for example greater than 40% relative to the total weight of the liquid fatty phase, or from 50% to 100%, of at least one apolar, such as hydrocarbon-based, oil. For the purposes of the invention, the expression “hydrocarbon-based oil” means an oil comprising carbon and hydrogen atoms, optionally with at least one group chosen from hydroxyl, ester, carboxyl and ether groups. With such a fatty phase, the at least one organogelator may, for example, contain an amine, amide, urea or urethane group.

For a liquid fatty phase structured with a polymer containing a partially silicone-based skeleton, this fatty phase may contain more than 30%, for example, more than 40%, relative to the total weight of the liquid fatty phase and, for example, from 50% to 100%, of at least one silicone-based liquid oil, relative to the total weight of the liquid fatty phase. In this embodiment, the at least one organogelator may comprise a silicone group.

For a liquid fatty phase structured with an apolar polymer of the hydrocarbon-based type, this fatty phase may contain more than 30%, for example more than 40% by weight, and, as a further example, from 50% to 100% by weight, of at least one liquid apolar, such as hydrocarbon-based, oil, relative to the total weight of the liquid fatty phase. In this embodiment, the at least one organogelator may contain hydrocarbon groups chosen from linear, branched and cyclic hydrocarbon-based groups, such as C₁ to C₄₀ groups.

For example, the at least one polar oil useful in the invention may be chosen from:

hydrocarbon-based plant oils with a high content of triglycerides comprising fatty acid esters of glycerol in which the fatty acids may have varied chain lengths from C₄ to C₂₄, these chains possibly being chosen from linear and branched, and saturated and unsaturated chains; these oils can be chosen from, for example, wheat germ oil, corn oil, sunflower oil, karite butter, castor oil, sweet almond oil, macadamia oil, apricot oil, soybean oil, cotton oil, alfalfa oil, poppy oil, pumpkin oil, sesame oil, marrow oil, rapeseed oil, avocado oil, hazelnut oil, grape seed oil, blackcurrant seed oil, evening primrose oil, millet oil, barley oil, quinoa oil, olive oil, rye oil, safflower oil, candlenut oil, passion flower oil and musk rose oil; or alternatively caprylic/capric acid triglycerides such as those sold by Stearineries Dubois or those sold under the names Miglyol 810, 812 and 818 by Dynamit Nobel;

synthetic oils or esters of formula R₅COOR₆ in which R₅ is chosen from linear and branched fatty acid residues containing from 1 to 40 carbon atoms and R₆ is chosen from, for example, a hydrocarbon-based chain containing from 1 to 40 carbon atoms, on condition that R₅+R₆≧10, such as, for example, purcellin oil (cetostearyl octanoate), isononyl isononanoate, C₁₂-C₁₅ alkyl benzoates, isopropyl myristate, 2-ethylhexyl palmitate, isostearyl isostearate and alkyl or polyalkyl octanoates, decanoates or ricinoleates; hydroxylated esters such as isostearyl lactate and diisostearyl malate; and pentaerythritol esters;

synthetic ethers containing from 10 to 40 carbon atoms;

C₈ to C₂₆ fatty alcohols such as oleyl alcohol; and

C₈ to C₂₆ fatty acids such as oleic acid, linolenic acid or linoleic acid;

and mixtures thereof.

The at least one apolar oil according to the invention is chosen from, for example, silicone oils chosen from volatile and non-volatile, linear and cyclic polydimethylsiloxanes (PDMSs) that are liquid at room temperature; polydimethylsiloxanes comprising alkyl or alkoxy groups which are pendant and/or at the end of the silicone chain, the groups each containing from 2 to 24 carbon atoms; phenylsilicones such as phenyl trimethicones, phenyl dimethicones, phenyl trimethylsiloxy diphenylsiloxanes, diphenyl dimethicones, diphenyl methyldiphenyl trisiloxanes and 2-phenylethyl trimethylsiloxysilicates; hydrocarbons chosen from linear and branched, volatile and non-volatile hydrocarbons of synthetic and mineral origin, such as volatile liquid paraffins, (such as isoparaffins and isododecane) or non-volatile liquid paraffins and derivatives thereof, liquid petrolatum, liquid lanolin, polydecenes, hydrogenated polyisobutene such as Parleam®, and squalane; and mixtures thereof. The structured oils, for example those structured with polyamides such as those of formula (I) or with polyurethanes, polyureas, polyurea-urethanes, in accordance with the invention, may be, in one embodiment, apolar oils, such as an oil or a mixture of hydrocarbon oils chosen from those of mineral and synthetic origin, chosen from hydrocarbons such as alkanes such as hydrogenated polybutene, e.g. Parleam® oil made or sold by Nippon Oil Fats, isoparaffins including isododecane, and squalane, and mixtures thereof. These oils may, in one embodiment, be combined with at least one phenylsilicone oil.

The liquid fatty phase, in one embodiment, contains at least one non-volatile oil chosen from, for example, hydrocarbon-based oils of mineral, plant and synthetic origin, synthetic esters or ethers, silicone oils and mixtures thereof.

For the purposes of the invention, the expression “volatile solvent or oil” means any non-aqueous medium capable of evaporating on contact with the skin or the lips in less than one hour at room temperature and atmospheric pressure. The volatile solvent(s) of the invention is(are) organic solvents, such as volatile cosmetic oils that are liquid at room temperature, having a non-zero vapor pressure, at room temperature and atmospheric pressure, ranging in particular from 10⁻² to 300 mmHg (1.33 to 40 000 Pa) and, for example, greater than 0.03 mm Hg (4 Pa) and even greater than 0.3 mmHg (40 Pa). The expression “non-volatile oil” means an oil which remains on the skin or the lips at room temperature and atmospheric pressure for at least several hours, such as those having a vapor pressure of less than 10⁻² mmHg (1.33 Pa).

According to the invention, these volatile solvents (or oils) may facilitate the staying power or long wearing properties of the composition on the skin, the lips or superficial body growths, such as keratinous fibers and nails. The solvents can be chosen from hydrocarbon-based solvents, silicone solvents optionally comprising alkyl or alkoxy groups that are pendant or at the end of a silicone chain, fluorinated solvents, and a mixture of these solvents.

The volatile oil(s), in one embodiment, is present in an amount ranging up to 95.5% relative to the total weight of the composition, such as from 2% to 75%, and, as a further example, from 10% to 45%. This amount will be adapted by a person skilled in the art according to the desired staying power or long wearing properties.

The at least one liquid fatty phase of the composition of the invention may further comprises a dispersion of lipid vesicles. The composition of the invention may also, for example, be in the form of a fluid anhydrous gel, a rigid anhydrous gel, a fluid simple emulsion, a fluid multiple emulsion, a rigid simple emulsion or a rigid multiple emulsion. The simple emulsion or multiple emulsion may comprise a continuous phase chosen from an aqueous phase optionally containing dispersed lipid vesicles, or a fatty phase optionally containing dispersed lipid vesicles. In one embodiment, the composition has a continuous oily phase or fatty phase and is more specifically an anhydrous composition, for example, a stick or dish form. An anhydrous composition is one that has less than 10% water by weight, such as, for example, less than 5% by weight.

Additional Rheological Agent

The composition of the invention may also comprise at least one additional rheological agent capable of further limiting any exudation of the composition when it is in stick form. The at least one additional rheological agent may be chosen from waxes, polymeric gelling agents and mineral gelling agents for the liquid fatty phase. The composition may, for example, contain at least one liposoluble or lipodispersible, polymeric or mineral gelling agent.

Liposoluble or Lipodispersible Gelling Agent

As mineral gelling agents which may be used, mention may be made of clays optionally modified with an ammonium chloride of a C₁₀ to C₂₂ fatty acid, for instance hectorite modified with distearyidimethylammonium chloride, such as the products sold or made under the names Bentone 34 by the company Rheox, and silicas, such as fumed silicas and hydrophobic silicas. As polymeric gelling agents, mention may be made of partially or totally crosslinked elastomeric polyorganosiloxanes of three-dimensional structure, such as the products sold or made under the names KSG6 from Shin-Etsu, Trefil E-505C or Trefil E-506C from Dow Corning, Gransil from Grant Industries (SR-CYC, SR DMF10, SR-DC556) or those sold or made in the form of preconstituted gels (KSG15, KSG17, KSG16, KSG18, KSG-21 from Shin-Etsu, Gransil SR 5CYC gel, Gransil SR DMF10 gel, Gransil SR DC556 gel, SF 1204 and JK 113 from General Electric); galactomannans containing 1 to 6, for example, from 2 to 4 hydroxyl groups per sugar, substituted with a saturated or unsaturated alkyl chain, for instance guar gum alkylated with C₁ to C₆, for example C₁ to C₃ alkyl chains, and such as ethylated guar gum having, for example, a degree of substitution of 2 to 3, as disclosed in document EP-A-708 114 and sold or made by the company Aqualon under the name N-Hance-AG 200® or N-Hance AG 50®; diblock or triblock polymers or copolymers, or even polymers or copolymers of multiblock or starburst or radial type resulting from the polymerization or copolymerization of an ethylenic monomer containing at least one ethylenic and preferably conjugated bond (or diene), in particular such as polystyrene/copoly(ethylene-butylene) or polystyrene/copoly(ethylene-propylene) such as those sold or made under the brand name “Kraton” by Shell Chemical Co. or Gelled Permethyl 99A by Penreco; silicone gums; ethylcellulose, for instance the products sold under the name Ethocel by Dow Chemical; mixtures thereof.

The additional rheological agent is, in one embodiment, hydrophobic-treated fumed silica having a particle size which can be nanometric to micrometric, for example ranging from about 5 mm to 200 mm. The hydrophobic groups may be:

trimethylsiloxyl groups, which are obtained, for example, by treating fumed silica in the presence of hexamethyldisilazane. Silicas thus treated are known as “silica silylate” according to the CTFA (6th edition, 1995). They are sold or made, for example, under the references “Aerosil R812®” by the company Degussa and “CAB-O-SIL TS-530®” by the company Cabot;

dimethylsilyloxyl or polydimethylsiloxane groups, which are obtained, for example, by treating fumed silica in the presence of polydimethylsiloxane or dimethyldichlorosilane. Silicas thus treated are known as “silica dimethyl silylate” according to the CTFA (6th edition, 1995). They are sold or made, for example, under the references “Aerosil R972®” and “Aerosil R974®” by the company Degussa, and “CAB-O-SIL TS-610®” and “CAB-O-SIL TS-720®” by the company Cabot;

groups derived from reacting fumed silica with silane alkoxides or siloxanes. These treated silicas are, for example, the products sold or made under the reference “Aerosil R805®” by the company Degussa.

The additional polymeric or mineral rheological agent(s) (i.e. gelling agents) may make it possible to limit the exudation of the composition and to increase its stability, while at the same time conserving the composition's glossy appearance, unlike waxes. The additional polymeric or mineral rheological agent(s) may be used, for example, at concentrations of from 0.05% to 35% relative to the total weight of the composition, for example from 0.5% to 20%, and, as a further example, from 1% to 10%, if present.

Waxes

The composition can optionally contain at least one wax to further limit the exudation of the composition in stick form, although this rigid form can be obtained in the absence of wax. For the purposes of the present invention, a wax is a lipophilic fatty compound that is solid at room temperature (25° C.) and atmospheric pressure (760 mmHg, i.e. 101 Kpa), which undergoes a reversible solid/liquid change of state, having a melting point of greater than 40° C., for example greater than 55° C. and which may be up to 200° C., and having an anisotropic crystal organization in the solid state. By bringing the wax to its melting point, it is possible to make it miscible with oils and to form a microscopically homogeneous mixture, but on returning the temperature of the mixture to room temperature, recrystallization of the wax in the oils of the mixture is obtained. The waxes may be present in an amount ranging up to 20% relative to the total weight of the composition, for example from 0.1% to 15%, and, as a further example, from 1% to 5%.

For the purposes of the invention, the waxes are those generally used in cosmetics and dermatology; such as those of natural origin, for instance beeswax, carnauba wax, candelilla wax, ouricury wax, Japan wax, cork fibre wax, sugar cane wax, paraffin wax, lignite wax, microcrystalline waxes, lanolin wax, montan wax, ozokerites and hydrogenated oils such as hydrogenated jojoba oil as well as waxes of synthetic origin, for instance polyethylene waxes derived from the polymerization of ethylene, waxes obtained by Fischer-Tropsch synthesis, fatty acid esters and glycerides that are solid at 40° C., for example, at above 55° C., silicone waxes such as alkyl- and alkoxy-poly(di)methylsiloxanes and/or poly(di)methylsiloxane esters that are solid at 40° C. and, for example, at above 55° C.

According to the invention, the melting point values correspond to the melting peak measured by the differential scanning calorimetry method with a temperature rise of 5 or 10° C./min.

Additional Additive

The composition of the invention can also comprise any additional additive usually used in the field under consideration, such as cosmetics or dermatology, chosen, for example, from antioxidants, essential oils, preserving agents, fragrances, fillers (Polytrap® from Dow Corning), fatty compounds that are pasty or viscous at room temperature, neutralizing agents, gums, liposoluble polymers or polymers that are dispersible in the medium, cosmetic or dermatological active agents such as, for example, emollients, moisturizers, vitamins, essential fatty acids, dispersants such as poly(12-hydroxystearic acid), coloring agents and mixtures thereof. These additives, if present, may each be present in the composition in a proportion of up to 20%, for example from 0.01% to 20%, and, as a further example, from 0.01% to 10% relative to the total weight of the composition. This water may be present in the composition from 0.01% to 50%, for example form 0.5 to 30% relative to the total weight of the composition. For an emulsion, the water can represent up to 50%.

The composition of the invention can also contain, as an additional additive, an aqueous phase containing water that is optionally thickened with an aqueous-phase thickener or gelled with a hydrophilic gelling agent and optionally water-miscible compounds. This water may be present in the composition from 0.01% to 50%, for example from 0.5 to 30% relative to the total weight of the composition. For an emulsion, the water can represent up to 50%.

Needless to say, a person skilled in the art will take care to select the optional additional additives and/or the amount thereof such that the advantageous properties of the composition according to the invention are not, or are not substantially, adversely affected by the envisaged addition.

The composition according to the invention can be in the form of a tinted or non-tinted dermatological composition or care composition for keratin materials such as the skin, the lips and/or superficial body growths, in the form of an antisun composition or body hygiene composition, such as in the form of a deodorant product or make-up-removing product in stick form. It can be used, for example, as a care base for the skin, superficial body growths, such as keratinous fibers, or the lips, for example, lip balms, for protecting the lips against cold and/or sunlight and/or the wind, or care cream for the skin, the nails or the hair.

The composition of the invention may also be in the form of a colored make-up product for the skin, such as a foundation, optionally having care or treating properties, a blusher, a face powder, an eyeshadow, a concealer product, an eyeliner, a make-up product for the body; a make-up product for the lips such as a lipstick, optionally having care or treating properties; a make-up product for superficial body growths, such as the nails or the eyelashes, for example in the form of a mascara cake, or for the eyebrows and the hair, for example in the form of a pencil.

Needless to say, the composition of the invention should be cosmetically or dermatologically acceptable, i.e., it should contain a non-toxic physiologically acceptable medium and should be able to be applied to the skin, superficial body growths or the lips of human beings. For the purposes of the invention, the expression “cosmetically acceptable” means a composition of pleasant appearance, odor, feel and taste.

The composition may also contain at least one cosmetic active agent and/or one dermatological active agent, i.e. an agent having a beneficial effect on the skin, lips or body growths, and/or at least one coloring agent.

In one embodiment, the composition does not contain sensibilizing agent as for example colophony.

Coloring Agent

The coloring agent according to-the invention may be chosen from the lipophilic dyes, hydrophilic dyes, pigments and nacreous pigments (i.e. nacres) usually used in cosmetic or dermatological compositions, and mixtures thereof. This coloring agent can generally be present in a proportion of from 0.01% to 50% relative to the total weight of the composition, for example from 0.5% to 40%, and, as a further example, from 5% to 30%, if it is present. In the case of a composition in the form of a free or compacted powder, the amount of coloring agent in the form of solid particles that are insoluble in the medium (nacres and/or pigments) may be up to 90% relative to the total weight of the composition.

The liposoluble dyes include, for example, Sudan Red, D&C Red 17, D&C Green 6, β-carotene, soybean oil, Sudan Brown, D&C Yellow 11, D&C Violet 2, D&C Orange 5, quinoline yellow and annatto. They can be present in an amount ranging from 0.1% to 20% of the weight of the composition, for example from 0.1% to 6% (if present). The water-soluble dyes are, for example, beetroot juice or methylene blue, and can represent from 0.01 to 6% of the total weight of the composition.

The pigments may be white or colored, mineral and/or organic, and coated or uncoated and having a micron size or not. Among the mineral pigments which may be mentioned are titanium dioxide, optionally surface-treated, zirconium oxide, zinc oxide or cerium oxide, as well as iron oxide, chromium oxide, manganese violet, ultramarine blue, chromium hydrate and ferric blue. Among the organic pigments which may be mentioned are carbon black, pigments of D&C type, and lakes based on cochineal carmine or on barium, strontium, calcium or aluminium. The pigment(s) can be present in an amount ranging from 0.1% to 50%, for example from 0.5% to 40%, and, as a further example, from 2% to 30% relative to the total weight of the composition, if they are present.

The nacreous pigments may be chosen from white nacreous pigments such as mica coated with titanium or with bismuth oxychloride, colored nacreous pigments such as titanium mica with iron oxides, titanium mica with, for example, ferric blue or chromium oxide, titanium mica with an organic pigment of the type mentioned above, as well as nacreous pigments based on bismuth oxychloride or alternatively interferential or goniochromatic pigments. They can be present in an amount ranging from 0.1% to 20% relative to the total weight of the composition, for example from 0.1% to 15%, if they are present.

In one embodiment, the coloring agent is chosen from pigments (nacreous or not).

Pasty Fatty Compound

The composition according to the invention may also contain at least one fatty compound that is pasty or viscous at room temperature. For the purposes of the invention, the expression “pasty fatty substance” means a fatty substance with a melting point ranging from 20 to 55° C., for example from 25 to 45° C., and as further example from 25 to 40° C. and/or a viscosity at 40° C. ranging from 0.1 to 40 Pa.s (1 to 400 poises), for example from 0.5 to 25 Pa.s, measured using a Contraves TV or Rheomat 80 viscometer, equipped with a spindle rotating at 240 min⁻¹ for supplying with 60 Hz, or at 200 min⁻¹ for supplying with 50 Hz. A person skilled in the art can select the spindle for measuring the viscosity from the spindles MS-r3 and MS-r4, on the basis of his general knowledge, so as to be able to carry out the measurement of the pasty compound tested.

Among the pasty compounds which may be used in the composition according to the invention, mention may be made of lanolins and lanolin derivatives such as acetylated lanolins or oxypropylenated lanolins or isopropyl lanolate, having a viscosity of from 18 to 21 Pa.s, for instance 19 to 20.5 Pa.s, and/or a melting point of from 30° C. to 55° C., and for example from 30° C. to 40° C., and mixtures thereof. It is also possible to use esters of fatty acids or of fatty alcohols, such as those containing from 20 to 65 carbon atoms (melting point of about from 20 to 35° C. and/or viscosity at 40° C. ranging from 0.1 to 40 Pa.s), such as triisostearyl citrate or cetyl citrate; arachidyl propionate; polyvinyl laurate; cholesterol esters, such as triglycerides of plant origin, such as hydrogenated plant oils (hydrogenated castor oil), viscous polyesters such as poly(12-hydroxystearic acid); polydimethylsiloxanes (PDMS) having alkyl or alkoxy pendant chains containing from 8 to 24 carbon atoms, and a melting point of 20-55° C., and for example from 20° C. to 40° C. such as stearyldimethicones (in particular DC2503 and DC25514 from Dow Corning); and mixtures thereof.

The pasty fatty substance(s) may be present in a proportion up to 60% by weight, relative to the total weight of the composition, for example from 0.1% to 45% by weight, and, as a further example, from 2% to 30% by weight, in the composition, if they are present.

The composition according to the invention may be manufactured by the known processes, that are generally used in cosmetics or dermatology. It may be manufactured by the process which comprises heating the polymer at least to its softening point, adding the organogelator, and optionally the amphiphilic compound(s), the coloring agents and the additives thereto and then mixing everything together until a clear, transparent solution is obtained. After reducing the temperature, the volatile solvent(s) is(are) then added to the mixture obtained. The homogeneous mixture obtained can then be cast in a suitable mold such as a lipstick mold or directly into the packaging articles (for example, a case or dish).

Another embodiment of the invention is a lipstick composition in stick form containing at least one continuous liquid fatty phase, at least one organogelator for the fatty phase and at least one non-waxy structuring polymer having a weight-average molecular mass of less than 100,000, the liquid fatty phase, the structuring polymer and the organogelator forming a physiologically acceptable medium. The organogelator and the structuring polymer may be such that they give the composition the appearance of a deformable elastic solid with a hardness ranging from 30 gf to 300 gf (294 N to 2 940 N), such as 30gf to 250 gf (294 N to 2 450 N), and further such as 30 gf to 200 gf (294 N to 1 960 N), even in the absence of wax, as measured by the “cheese wire” method discussed above.

The non-waxy polymer is, in one embodiment, a polymer whose skeleton comprises hydrocarbon-based units containing a hetero atom, as defined previously, and is, for example, a polyamide group that may contain alkyl end groups linked to the skeleton via a linking group, such as of the ester type.

This lipstick contains, for example, at least one additive chosen from fatty compounds that are pasty at room temperature, waxes and fillers, and mixtures thereof.

An aspect of the invention is also a care, make-up or treatment cosmetic process for keratin materials of human beings, such as superficial body growths, such as keratinous fibers, the skin, the lips, comprising the application to the keratin materials of the composition, for example the cosmetic composition, as defined above.

Another aspect of the invention is the use of the composition of the invention, discussed above, for the manufacture of a physiologically acceptable composition, the combination serving to give the said composition at least one of the following: a solid appearance, in particular without wax, and/or non-exudation and shear-strength properties and/or properties of producing a glossy and/or comfortable deposit on keratin materials.

Another aspect of the invention is a cosmetic process for limiting the exudation of a fatty liquid phase of a composition cast in particular as a stick, the said composition containing at least one structuring polymer comprising a) a polymer skeleton comprising at least one hydrocarbon-based repeating unit comprising at least one hetero atom, and b) optionally at least one terminal fatty chain, optionally functionalized, comprising at least one chain chosen from alkyl and alkenyl chains, such as alkyl and alkenyl chains having at least 4 carbon atoms, and further such as alkyl and alkenyl chains having from 8 to 120 carbon atoms, bonded to the polymer skeleton via at least one linking group, and c) optionally at least one pendant fatty chain, optionally functionalized, comprising at least one chain chosen from alkyl and alkenyl chains, such as alkyl and alkenyl chains having at least 4 carbon atoms, and further such as alkyl and alkenyl chains having from 8 to 120 carbon atoms, bonded to the polymer skeleton via at least one linking group, and of at least one organogelator, which comprises introducing a sufficient amount of at least one organogelator into the composition.

Another aspect of the invention is a composition comprising at least one liquid fatty phase which comprises:

(i) at least one structuring polymer comprising:

a polymer skeleton which comprises a) at least one hydrocarbon-based repeating unit comprising at least one hetero atom and b) at least one of:

at least one terminal fatty chain, optionally functionalized, chosen from alkyl chains and alkenyl chains, wherein said at least one terminal fatty chain is bonded to said polymer skeleton via at least one linking group; and

at least one pendant fatty chain, optionally functionalized, chosen from alkyl chains and alkenyl chains, wherein said at least one pendant fatty chain is bonded to said polymer skeleton via at least one linking group; and

(ii) at least one organogelator.

The invention is illustrated in greater detail in the examples which follow. The amounts are given as percentages by mass.

Counter Example 1: Lipstick

| Phase A | |
|---|---|
| Uniclear 100 | 18% |
| Isononyl isononanoate | 5% |
| Diisostearyl malate | 17% |
| Hydrogenated polybutene (Parleam) | 4% |
| Phase B | |
| Hydrophobic fumed silica (Aerosil R972) | 3% |
| Hydrogenated polybutene (Parleam) | 25% |
| Isononyl isononanoate | 12% |
| Phase C | |
| Pigments | 7% |
| Hydrogenated polybutene (Parleam) | 9% |

Procedure

The Uniclear 100 and the oils of phase A were introduced into a heating vessel. The mixture was placed under magnetic stirring and then heated in a first stage to 100° C. (to liquefy the Uniclear). A mixture comprising the silica gel (phase B), prepared beforehand, and the ground pigmentary material (phase C), which was heated beforehand to 100° C. and homogenized with stirring, was introduced. The product obtained was placed in a heated mold (T°=45° C.) with stirring and, once setting had begun, was placed in a freezer (T°=−21° C.) for 15 minutes.

a) Silica Gel (Phase B)

The gel was prepared, with stirring, in a Rayneri stirrer at 60° C., using a hot plate, by introducing the silica portionwise into the oily mixture formed from:

| | |
|---|---|
| Hydrogenated polybutene (Parleam) | 25 g |
| Isononyl isononanoate | 12 g |
| TOTAL | 40 g |

b) Ground Pigmentary Material (Phase C)

The pigments (iron oxide+titanium oxide) were mixed with the oil heated to 60° C.; the mixture was ground 3 times in a three-roll mill.

The sticks of lipstick had a diameter of 12.7 mm and a hardness of 77±10 gf (754 N±98 N) measured using a “cheese wire”. These sticks of lipstick broke during the measurement of the dynamic fragility carried out on 3 sticks. The fragility of the composition is determined by a method wherein the stick is submitted to several back-and-forth movements on a support for 3 minutes at a speed of 60 back-and-forth movements/minute, at 20° C. The result is defined by the number of broken sticks with respect to the number of tested sticks.

### EXAMPLE 1

### Lipstick

| Phase A | |
|---|---|
| Uniclear 100 | 18% |
| GP-1 from Ajinomoto | 5% |
| Isononyl isononanoate | 3.33% |
| Diisostearyl malate | 15.33% |
| Hydrogenated polybutene (Parleam) | 2.34% |
| Phase B | |
| Hydrophobic silica | 3% |
| Hydrogenated polybutene (Parleam) | 25% |
| Isononyl isononanoate | 12% |
| Phase C | |
| Pigments | 7% |
| Hydrogenated polybutene (Parleam) | 9% |

Procedure

The Uniclear 100, the GP-1 and the oils of phase A were introduced into a heating vessel. The mixture was placed under magnetic stirring and heated in a first stage to 100° C. (to liquefy the Uniclear) and heating was then continued to the temperature required to obtain a transparent homogeneous liquid. The mixture was then placed 10° C. above this temperature. Next, a mixture comprising the silica gel (phase B), prepared beforehand, and the ground pigmentary material (phase C), which was heated beforehand to 100° C. and homogenized with magnetic stirring, was introduced.

The mixture obtained was left stirring for one hour and the composition was then cast in a mold (T°=45° C.) which was placed, after setting had begun, in a freezer (T°=−21° C.) for 15 minutes.

The silica gel and the ground pigmentary material were prepared as in Counterexample 1.

The sticks of lipstick obtained had a diameter of 12.7 mm and a hardness of 204±20 gf (1 999 N±196 N) measured using a “cheese wire”. These sticks of lipstick did not break during measurement of the dynamic fragility carried out on 3 sticks.

### EXAMPLE 2

### Lipstick

| Phase A | |
|---|---|
| Uniclear 100 | 18% |
| Trans-N, N′-bis(dodecanoyl)-1,2-diaminocyclohexane* | 5% |
| Isononyl isononanoate | 3.33% |
| Diisostearyl malate | 15.33% |
| Hydrogenated polybutene (Parleam) | 2.34% |
| Phase B | |
| Hydrophobic fumed silica | 3% |
| Hydrogenated polybutene (Parleam) | 25% |
| Isononyl isononanoate | 12% |
| Phase C | |
| Pigments | 7% |
| Hydrogenated polybutene (Parleam) | 9% |

Compound of formula (II) with A=1,2 cyclohexylene and R═R′ and being a linear chain containing 11 carbon atoms, called hereafter trans-diaminocyclohexane derivative, resulting from the reaction to the trans-1,2 diaminocyclohexane with the lauroyl chloride with the ration 30/60.

Procedure

The Uniclear 100, the trans-diaminocyclohexane derivative and the oils of phase A were introduced into a heating vessel. Phase A obtained was placed under magnetic stirring and heated in a first stage to 100° C. (to liquefy the Uniclear) and heating was then continued to the temperature required to obtain a transparent homogeneous liquid. The mixture was then placed 10° C. above this temperature. Next, a mixture comprising the silica gel (phase B), prepared beforehand, and the ground pigmentary material (phase C), which was heated beforehand to 100° C. and homogenized with magnetic stirring, was introduced.

The mixture obtained was left stirring for one hour and the composition was then cast in a mold (T°=45° C.) which was placed, after setting had begun, in a freezer (T°=−21° C.) for 15 minutes.

The silica gel and the ground pigmentary material were prepared as in Counterexample 1.

The sticks of lipstick obtained had a diameter of 12.7 mm and a hardness of 180±20 gf (1 764 N±196 N) measured using a “cheese wire”. These sticks of lipstick did not break during measurement of the dynamic fragility carried out on 3 sticks. They did not exude and they deposited a glossy make-up on the lips.

### EXAMPLE 3

### Lipstick

| Phase A | |
|---|---|
| Uniclear 100 | 18 g |
| GP-1 (Ajinomoto) | 5 g |
| Polyethylene wax (PMw * 500) | 3 g |
| Liquid lanolin | 5 g |
| BHT | 0.07 g |
| Octyl dodecanol | 8.25 g |
| Phenyl silicone (20 cSt at 25° C.) | 4.58 g |
| Hydrogenated polybutene (Parleam) | 22.24 g |
| Phase B | |
| Polytrap ® (Dow Corning) | 3 g |
| Hydrogenated polybutene (Parleam) | 20 g |
| Pigments | 8.66 g |
| Phase C | |
| Fragrance | 0.2 g |

* PMw is the weight-average molecular mass.

Procedure

Phase A was introduced into a heating vessel and placed under magnetic stirring. It was heated to the temperature required to obtain a transparent homogeneous liquid. The mixture was then placed 10° C. above this temperature.

When phase A was homogeneous, the pigments (phase B), ground in a three-roll mill, and then phase C were introduced. The mixture was then left stirring for 1 hour 30 minutes, after which the preparation was cast in a mold (T°=45° C.) which was placed, after setting had begun, in a freezer (T°=−21° C.) to carry out tempering.

The sticks of lipstick obtained had a diameter of 12.7 mm and a hardness of 204±20 gf (1 999 N±196 N) measured using a “cheese wire”. These sticks of lipstick did not break during measurement of the dynamic fragility carried out on 3 sticks. The deposit on the lips was comfortable and glossy.

### EXAMPLE 4

### Lipstick

| Phase A | |
|---|---|
| Uniclear 100 | 18 g |
| Trans-N, N′-bis (dodecanoyl)-1,2diaminocyclohexane | 5 g |
| Polyethylene wax (PMw 500) | 3 g |
| Liquid lanolin | 5 g |
| BHT | 0.07 g |
| Octyl dodecanol | 8.25 g |
| Phenyl silicone (20 cSt at 25° C.) | 4.58 g |
| Hydrogenated polybutene (Parleam) | 22.24 g |
| Phase B | |
| Polytrap ® | 3 g |
| Hydrogenated polybutene (Parleam) | 20 g |
| Pigments | 8.66 g |
| Phase C | |
| Fragrance | 0.2 g |

Procedure

The Uniclear 100 and the oils of phase A were introduced into a heating vessel. The mixture was placed under magnetic stirring. It was heated in a first stage to the temperature required to obtain a transparent homogeneous liquid. The mixture was then placed 10° C. above this temperature. When phase A was homogeneous, the pigments (phase B), ground in a three-roll mill, and then phase C were introduced. The mixture was then left stirring for 1 hour 30 minutes, after which the preparation was cast in a mold (T°=45° C.) which was placed, after setting had begun, in a freezer to carry out tempering.

The sticks of lipstick had a diameter of 12.7 mm and a hardness of 158±20 gf (1 548 N±196 N), measured using a “cheese wire”. These sticks of lipstick were glossy, did not exude and did not break during measurement of the dynamic fragility, carried out on 3 sticks.

What is claimed is:

1. An anhydrous composition comprising at least one liquid fatty phase which comprises:
(i) at least one structuring polymer comprising:
a polymer skeleton which comprises at least one hydrocarbon-based repeating unit comprising at least one hetero atom; and
(ii) at least one organogelator.

2. A composition comprising at least one liquid fatty phase which comprises:
(i) at least one structuring polymer comprising:
a polymer skeleton which comprises at least three hydrocarbon-based repeating units comprising at least one hetero atom; and
(ii) at least one organogelator.

3. A structured composition comprising at least one liquid fatty phase which comprises:
(i) at least one structuring polymer comprising:
a polymer skeleton which comprises at least one hydrocarbon-based repeating unit comprising at least one hetero atom; and
(ii) at least one organogelator, wherein said organogelator is not methyl- 12-hydroxystearate.

4. The composition according to claim 2 or 3, wherein the composition is in a form chosen from a fluid anhydrous gel, rigid anhydrous gel, fluid simple emulsion, rigid simple emulsion, fluid multiple emulsion, and rigid multiple emulsion.

5. The composition according to one of claims 1 to 4, wherein said at least one structuring polymer further comprises at least one of:
at least one terminal fatty chain chosen from alkyl chains and alkenyl chains, wherein said at least one terminal fatty chain is bonded to said polymer skeleton via at least one linking group; and
at least one pendant fatty chain chosen from alkyl chains and alkenyl chains, wherein said at least one pendant fatty chain is bonded to said polymer skeleton via at least one linking group.

6. The composition according to claim 5, wherein said alkyl chains and said alkenyl chains each comprise at least four carbon atoms.

7. The composition according to one of claims 5 to 6, wherein said alkyl chains and said alkenyl chains each comprise from 8 to 120 carbon atoms.

8. The composition according to one of claims 5 to 7, wherein said alkyl chains and said alkenyl chains each comprise from 12 to 68 carbon atoms.

9. The composition according to one of claims 5 to 8, wherein said at least one linking group is chosen from single bonds and urea, urethane, thiourea, thiourethane, thioether, thioester, ester, ether and amine groups.

10. The composition according to one of claims 5 to 9, wherein said at least one linking group is chosen from urea, ester, and amine groups.

11. The composition according to one of claims 5 to 10, wherein said at least one linking group is chosen from ester and amine groups.

12. The composition according to one of claims 5 to 11, wherein said at least one linking group is an ester group present in a proportion ranging from 15% to 40% of the total number of all ester and hetero atom groups in the at least one structuring polymer.

13. The anhydrous composition according to one of claims 5 to 12, wherein said at least one linking group is an ester group present in a proportion ranging from 20% to 35% of the total number of all ester and hetero atom groups in the at least one structuring polymer.

14. The composition according to one of claims 5 to 13, wherein said at least one terminal fatty chain is functionalized.

15. The composition according to one of claims 5 to 14, wherein said at least one pendant fatty chain is functionalized.

16. The composition according to one of claims 5 to 15, wherein in said at least one structuring polymer, the percentage of the total number of fatty chains ranges from 40% to 98% relative to the total number of all repeating units and fatty chains in the at least one structuring polymer.

17. The composition according to one of claims 5 to 16, wherein in said at least one structuring polymer, the percentage of the total number of fatty chains ranges from 50% to 95% relative to the total number of all repeating units and fatty chains in the at least one structuring polymer.

18. The composition according to one of claims 1 to 17, wherein said at least one structuring polymer has a weight-average molecular mass of less than 100,000.

19. The composition according to one of claims 1 to 18, wherein said at least one structuring polymer has a weight-average molecular mass of less than 50,000.

20. The composition according to one of claims 1 to 19, wherein said at least one structuring polymer has a weight-average molecular mass ranging from 1000 to 30,000.

21. The composition according to one of claims 1 to 20, wherein said at least one hydrocarbon based repeating unit comprises from 2 to 80 carbon atoms.

22. The composition according to one of claims 1 to 21, wherein said at least one hetero atom of said at least one hydrocarbon-based repeating unit is chosen from nitrogen, sulphur, and phosphorus.

23. The composition according to claim 22, wherein said at least one hetero atom is a nitrogen atom.

24. The composition according to one of claims 1 to 23, wherein said at least one hetero atom is combined with at least one atom chosen from oxygen and carbon to form a hetero atom group.

25. The composition according to claim 24, wherein said at least one hetero atom group is chosen from amide groups, carbamate groups, and urea groups.

26. The composition according to claim 24 or 25, wherein said at least one hetero atom group is an amide group and said polymer skeleton is a polyamide skeleton.

27. The composition according to claim 24 or 25, wherein said at least one hetero atom group is chosen from carbamate groups and urea groups and said polymer skeleton is chosen from polyurethane skeletons, polyurea skeletons, and polyurethane-polyurea skeletons.

28. The composition according to one of claims 1 to 26, wherein said at least one structuring polymer is chosen from polyamide polymers of formula (I): 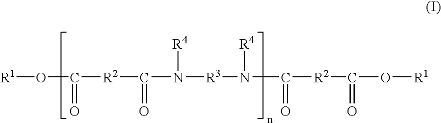
in which:
n is an integer which represents the number of amide units such that the number of ester groups present in said at least one polyamide polymer ranges from 10% to 50% of the total number of all ester groups and all amide groups comprised in said at least one polyamide polymer;
R¹, which are identical or different, are each chosen from alkyl groups comprising at least 4 carbon atoms and alkenyl groups comprising at least 4 carbon atoms;
R², which are identical or different, are each chosen from C₄ to C₄₂ hydrocarbon-based groups with the proviso that at least 50% of all R² are chosen from C₃₀ to C₄₂ hydrocarbon-based groups;
R³, which are identical or different, are each chosen from organic groups comprising atoms chosen from carbon atoms, hydrogen atoms, oxygen atoms and nitrogen atoms with the proviso that R³ comprises at least 2 carbon atoms; and
R⁴, which are identical or different, are each chosen from hydrogen atoms, C₁ to C₁₀ alkyl groups and a direct bond to at least one group chosen from R³ and another R⁴ such that when said at least one group is chosen from another R⁴, the nitrogen atom to which both R³ and R⁴ are bonded forms part of a heterocyclic structure defined in part by R⁴—N—R³, with the proviso that at least 50% of all R⁴ are chosen from hydrogen atoms.

29. The composition according to claim 28, wherein in said formula (I), n is an integer ranging from 1 to 5.

30. The composition according to claim 28 or 29, wherein in said formula (I), said alkyl groups of R¹ and said alkenyl groups of R¹ each independently comprise from 4 to 24 carbon atoms.

31. The composition according to one of claims 28 to 30, wherein in said formula (I), R¹, which are identical or different, are each chosen from C₁₂ to C₂₂ alkyl groups.

32. The composition according to one of claims 28 to 31, wherein in said formula (I), R¹, which are identical or different, are each chosen from C₁₆ to C₂₂ alkyl groups.

33. The anhydrous composition according to one of claims 28 to 32, wherein in said formula (I), R², which are identical or different, are each chosen from C₁₀ to C₄₂ hydrocarbon based groups with the proviso that at least 50% of all R² are chosen from C₃₀ to C₄₂ hydrocarbon based groups.

34. The composition according to one of claims 28 to 33, wherein in said formula (I), R³, which can be identical or different, are each chosen from C₂ to C₃₆ hydrocarbon-based groups and polyoxyalkylene groups.

35. The composition according to one of claims 28 to 34, wherein R³, which can be identical or different, are each chosen from C₂ to C₁₂ hydrocarbon-based groups.

36. The composition according to one of claims 28 to 35, wherein in said formula (I), R⁴, which can be identical or different, are each chosen from hydrogen atoms.

37. The composition according to one of claims 28 to 36, wherein said at least one polymer of formula (I) is in the form of a mixture of polymers, wherein said mixture optionally also comprises a compound of formula (I) wherein n is equal to zero.

38. The composition according to one of claims 1 to 37, wherein said at least one structuring polymer has a softening point greater than 50° C.

39. The composition according to one of claims 1 to 38, wherein said at least one structuring polymer has a softening point less than 150° C.

40. The composition according to one of claims 1 to 39, wherein said at least one structuring polymer has a softening point ranging from 70° C. to 130° C.

41. The composition according to one of claims 1 to 40, wherein said at least one structuring polymer is present in the composition in an amount ranging from 0.5% to 80% by weight relative to the total weight of the composition.

42. The composition according to one of claims 1 to 41, wherein said at least one structuring polymer is present in the composition in an amount ranging from 2% to 60% by weight relative to the total weight of the composition.

43. The composition according to one of claims 1 to 42, wherein said composition has a hardness ranging from 30 to 300 gf (294 N to 2 940 N).

44. The composition according to one of claims 1 to 43, wherein said composition has a hardness ranging from 30 to 250 gf (294 N to 2 450 N).

45. The composition according to one of claims 1 to 44, wherein said at least one liquid fatty phase of the composition further comprises at least one oil which is chosen from at least one polar oil and at least one apolar oil having an affinity with said at least one structuring polymer and/or with said at least one organogelator.

46. The composition according to claim 45, wherein said at least one polar oil is chosen from:
hydrocarbon-based plant oils with a high content of triglycerides comprising fatty acid esters of glycerol in which the fatty acids comprise chains having from 4 to 24 carbon atoms, said chains possibly being chosen from linear and branched, and saturated and unsaturated chains;
synthetic oils or esters of formula R₅COOR₆ in which R₅ is chosen from linear and branched fatty acid residues comprising from 1 to 40 carbon atoms and R₆ is chosen from hydrocarbon based chain containing from 1 to 40 carbon atoms with the proviso that R₅+R₆≧10;
synthetic ethers comprising from 10 to 40 carbon atoms;
C₈ to C₂₆ fatty alcohols; and
C₈ to C₂₆ fatty acids.

47. The composition according to claim 45, wherein said at least one apolar oil is chosen from:
silicone oils chosen from volatile and non-volatile, linear and cyclic polydimethylsiloxanes that are liquid at room temperature;
polydimethylsiloxanes comprising alkyl or alkoxy groups which are pendant and/or at the end of the silicone chain, the groups each comprising from 2 to 24 carbon atoms;
phenylsilicones; and
hydrocarbons chosen from linear and branched, volatile and non-volatile hydrocarbons of synthetic and mineral origin.

48. The composition according to one of claims 1 to 47, wherein said at least one liquid fatty phase further comprises at least one non-volatile oil.

49. The composition according to one of claims 1 to 48, wherein said at least one non-volatile oil is chosen from hydrocarbon-based oils of mineral, plant and synthetic origin, synthetic esters and ethers, and silicone oils.

50. The composition according to one of claims 1 to 49, wherein said at least one liquid fatty phase is present in an amount ranging from 1% to 99% by weight relative to the total weight of the composition.

51. The composition according to one of claims 1 to 50, wherein said at least one liquid fatty phase is present in an amount ranging from 10% to 80% by weight relative to the total weight of the composition.

52. The composition according to one of claims 1 to 51, wherein said at least one liquid fatty phase comprises at least one volatile solvent chosen from hydrocarbon-based solvents and silicone solvents optionally comprising at least one group chosen from alkyl and alkoxy groups that are pendant and/or at the end of a silicone chain.

53. The composition according to one of claims 1 to 52, wherein said composition further comprises at least one additional fatty material is chosen from gums, fatty materials pasty at ambient temperature, and resins.

54. The composition according to one of claims 1 to 53, wherein said at least one organogelator is chosen from non-polymeric organic compounds whose molecules are capable of, establishing, between themselves, at least one physical interaction leading to a self-aggregation of said molecules with formation of a macromolecular 3-dimensional network.

55. The composition according to claim 54, wherein said at least one physical interaction is chosen from self-complementary hydrogen interactions, interactions between unsaturated rings, dipolar interactions, and coordination bonding with organometallic derivatives.

56. The composition according to one of claims 1 to 55, wherein said at least one organogelator is chosen from compounds whose molecules comprise at least one entity chosen from at least one group capable of establishing hydrogen bonding; at least one aromatic ring; at least one bond comprising ethylenic unsaturation; and at least one asymmetric carbon.

57. The composition according to one of claims 1 to 56, wherein said at least one organogelator is a compound whose molecules comprise at least two groups capable of establishing hydrogen bonding.

58. The composition according to claim 57, wherein said at least one group capable of establishing hydrogen bonding is chosen from hydroxyl, carbonyl, amine, carboxylic acid, amide and benzyl groups.

59. The composition according to one of claims 1 to 58, wherein said at least one organogelator is chosen from:
hydroxylated carboxylic fatty acids comprising a chain chosen from linear and branched aliphatic carbon chains and salts thereof chosen from alkali metal and alkaline earth metal salts and esters thereof;
carboxylic acid amides;
amino acid amides and esters;
N-acylamino acid amides;
diamides having hydrocarbon-based chains, each containing from 1 to 22 carbon atoms, optionally substituted with at least one substituent chosen from ester, urea and fluoro groups;
steroid amines and amides and salts thereof;
compounds comprising several aromatic rings;
azobenzene steroids;
organometallic compounds;
surfactants in salt form comprising at least two chains chosen from linear and branched alkyl chains;
benzylidene sorbitols and alditols and derivatives thereof;
cyclodipeptides which are cyclic condensates of two amino acids;
cyclic compounds and alkylene compounds comprising two urea or urethane groups;
alkylaryl cyclohexanol derivatives;
callixarenes;
associations of 2,4,6-tri-aminopyrimidine substituted by an alkyl chain and dialkyl barbituric acid,
gluconamides derivatives,
bis oyxalyl amides of aminoacides,
amide and urea derivatives of lysine ester,
derivatives from benzene diamides of dicarboxylic acid,
monoalkyloxamides,
bola-amphiphile with 1 -glucosamide head,
bola-amphiphile amide derivatives,
alkyl-2-amonium-2-isobutylacetate p-toluene sulfonate
cellobiose fatty esters
diamides with terminal hydrocaron-based chain having 6 to 60 carbon atoms.

60. The composition according to claim 59, wherein in said hydroxylated carboxylic fatty acids, said chain comprises a carbon chain having at least 8 carbon atoms.

61. The composition according to claim 59, wherein said carboxylic acid amides are chosen from tricarboxylic acid amides.

62. The composition according to claim 61, wherein said tricarboxylic acid amides are chosen from cyclohexanetricarboxamides.

63. The composition according to claim 59, wherein said N-acylamino acid amides are chosen from diamides resulting from the action of an N-acylamino acid with an amine comprising from 1 to 22 carbon atoms.

64. The composition according to claim 59, wherein said hydrocabon-based chains of said diamides having hydrocarbon-based chains comprising from 1 to 22 carbon atoms contain from 6 to 18 carbon atoms.

65. The composition according to claims one of 1 to 59, wherein said at least one organogelator is chosen from N-acylamino acid amides, cyclohexane tricarboxamides and diamines having hydrocarbon-based chains, each chain containing from 1 to 22 carbon atoms, optionally substituted with at least one substituent chosen from ester, urea and fluoro groups;

66. The composition according to one of claims 1 to 59, wherein said at least one organogelator is chosen from compounds of formula (II) below:
R—CO—NH-A-NH—CO—R′
in which:
R and R′, which may be identical or different, are each chosen from a hydrogen atom and hydrocarbon-based chains chosen from saturated linear, saturated branched, saturated cyclic, unsaturated linear, unsaturated branched and unsaturated cyclic hydrocarbon-based chains comprising from 1 to 22 carbon atoms, said hydrocarbon-based chains being optionally substituted with at least one group chosen from an aryl —C₆H₅, an ester —COOR″, an amide —CONHR″, a urethane —OCONHR″ and a urea —NHCONHR″, wherein R″ is an alkyl group comprising from 2 to 12 carbon atoms; and/or said hydrocarbon-based chains optionally comprise from 1 to 3 hetero atoms chosen from O, S and N; and/or said hydrocarbon-based chains optionally are substituted with from 1 to 4 halogen atoms and/or with from 1 to 3 hydroxyl radicals, with the proviso that at least one of R and R′ is other than hydrogen; and
A is chosen from saturated and unsaturated, linear, cyclic and branched hydrocarbon-based chains comprising from 1 to 18 carbon atoms, optionally substituted with at least one group chosen from an aryl —C₆H₅, an ester —COOR″, an amide —CONHR″, a urethane —OCONHR″ and a urea —NHCONHR″ wherein R″ is an alkyl comprising from 2 to 12 carbon atoms; and/or optionally comprising from 1 to 3 hetero atoms chosen from O, S and N; and/or optionally substituted with from 1 to 4 halogen atoms and/or with from 1 to 3 hydroxyl radicals.

67. The composition according to one of claims 1 to 59, wherein said at least one organogelator is chosen from:
N,N′-bis(dodecanoyl)-1,2-diaminocyclohexane,
N,N′-bis(dodecanoyl)-1,3-diaminocyclohexane,
N,N′-bis(dodecanoyl)- 1,4-diaminocyclohexane,
N,N′-bis(dodecanoyl)-1,2-ethylenediamine,
N,N′-bis(dodecanoyl)-1-methyl-1,2-ethylenediamine,
N,N′-bis(dodecanoyl)-1,3-diaminopropane,
N,N′-bis(dodecanoyl)-1,12-diaminododecane,
N,N′-bis(dodecanoyl)-3,4-diaminotoluene,

68. The composition according to one of claims 1 to 59, wherein said at least one organogelator is chosen from compounds of formula (III): 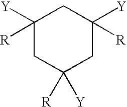
in which:
R is identical or different and each is chosen from a hydrogen atom, saturated linear hydrocarbon-based chains, and saturated branched hydrocarbon-based chains, wherein said hydrocarbon-based chains comprise from 1 to 6 carbon atoms;
Y is identical or different and each is chosen from the following groups:
—CO—S—R′; —CO—NHR′; —NH—COR′ and —S—COR′; in which R′ is identical or different and each is chosen from:
a hydrogen atom,
aryl groups,
aralkyl groups, and
saturated hydrocarbon-based chains chosen from linear, branched and cyclic hydrocarbon-based chains comprising from 1 to 22 carbon atoms, optionally substituted with at least one group chosen from aryl, ester, amide and urethane groups; and/or optionally comprising at least one hetero atom chosen from O, S and N; and/or optionally substituted with at least one fluorine atom and/or hydroxyl radical.

69. The composition according to claim 68, wherein in said formula (III), each R is a hydrogen atom.

70. The composition according to claim 68 or 69, wherein in said formula (III), each Y is chosen from the groups —CO—NHR′ and —NH—COR′.

71. The composition according to one of claims 68 to 70, wherein in said formula (III), R′ is chosen from aryl groups; aralkyl groups, wherein the alkyl portion is chosen from linear and branched alkyl chains comprising 12-16 carbon atoms; and linear and branched C₁₂-C₁₈ alkyl chains.

72. The composition according to one of claims 68 to 71, wherein said at least one organogelator is chosen from:
cis-1,3,5-tris(dodecylaminocarbonyl)cyclohexane,
cis-1,3,5-tris(octadecylaminocarbonyl)cyclohexane,
cis-1,3,5-tris[N-(3,7-dimethyloctyl)-aminocarbonyl]cyclohexane,
trans-1,3,5-trimethyl-1,3,5-tris(dodecylaminocarbonyl)cyclohexane, and
trans-1,3,5-trimethyl-1,3,5-tris(octadecylaminocarbonyl)cyclohexane.

73. The composition according to one of claims 1 to 72, wherein said at least one organogelator is present in an amount ranging from 0.1% to 80% by weight relative to the total weight of the composition.

74. The composition according to one of claims 1 to 73, wherein said at least one organogelator is present in an amount ranging from 0.5% to 60% by weight relative to the total weight of the composition.

75. The composition according to one of claims 1 to 74, wherein said composition is a solid.

76. The composition according to one of claims 1 to 75, wherein said composition is a solid chosen from molded and poured sticks.

77. The composition according to one of claims 1 to 76, wherein said at least one organogelator and/or said at least one structuring polymer have an affinity with a chemical portion of lone of the oils forming the liquid fatty phase of the composition so that hydrogen bonds with the oils are formed.

78. The composition according to one of claims 1 to 77, further comprising at least one amphiphilic compound that is liquid and non-volatile at room temperature and has a hydrophilic/lipophilic balance value of less than 12.

79. The composition according to claim 78, wherein said at least one amphiphilic compound comprises a lipophilic part linked to a polar part, the lipophilic part comprising a carbon-based chain comprising at least 8 carbon atoms.

80. The composition according to one of claims 78 or 79, wherein said at least one amphiphilic compound is present in an amount ranging from 0.1% to 35% by weight relative to the total weight of the composition.

81. The composition according to one of claims 78 to 80, wherein said at least one amphiphilic compound is present in an amount ranging from 1% to 20% by weight relative to the total weight of the composition.

82. The composition according to one of claims 1 to 81, further comprising at least one additional Theological agent.

83. The composition according to claim 82, wherein said at least one additional rheological agent is chosen from waxes, polymeric gelling agents and mineral gelling agents for the: liquid fatty phase.

84. The composition according to one of claims 1 to 83, further comprising at least one additional additive chosen from antioxidants, essential oils, preserving agents, fragrances, fillers, fatty compounds that are pasty at room temperature, neutralizing agents, gums, liposoluble polymers and polymers that are dispersible in a lipophilic medium, cosmetic and dermatological active agents, dispersants, and an aqueous phase comprising water that is optionally thickened or gelled with an aqueous-phase thickener or gelling agent and optionally water-miscible compounds.

85. The composition according to one of claims 1 to 84, further comprising at least one coloring agent.

86. The composition according to claim 85, wherein said at least one coloring agent is chosen from pigments.

87. The composition according to claims 85 or 86, wherein said at least one coloring agent is present in a proportion of from 0.01% to 50% relative to the total weight of the composition.

88. The composition according to one of claims 1 to 87, wherein said composition is in the form of a rigid gel.

89. The composition according to one of claims 1 to 88, wherein said composition is in the form of an anhydrous stick.

90. The composition according to one of claims 1 to 89, wherein said composition further comprises at least one wax.

91. The composition according to claim 90, wherein said at least one wax is chosen from beeswax, carnauba wax, candelilla wax, ouricury wax, Japan wax, cork fiber wax, sugar cane wax, paraffin wax, lignite wax, microcrystalline waxes, lanolin wax, montan wax, ozokerites and hydrogenated oils, polyethylene waxes, waxes obtained by Fischer-Tropsch synthesis, fatty acid esters and glycerides that are solid at 40° C., and silicone waxes.

92. An anhydrous composition comprising at least one liquid fatty phase which comprises:
(i) at least one structuring polymer, wherein said at least one structuring polymer is at least one polyamide polymer comprising:
a polymer skeleton which comprises at least one amide repeating unit; and
(ii) at least one organogelator.

93. The anhydrous composition according to claim 92, wherein said at least one polyamide polymer is chosen from polymers resulting from at least one polycondensation reaction between at least one acid chosen from dicarboxylic acids comprising at least 32 carbon atoms and at least one amine chosen from diamines comprising at least 2 carbon atoms and triamines comprising at least 2 carbon atoms.

94. The anhydrous composition according to claim 93, wherein said dicarboxylic acids comprise from 32 to 44 carbon atoms and said amines comprise from 2 to 36 carbon atoms.

95. The anhydrous composition according to claims 93 or 94, wherein said dicarboxylic acids are chosen from dimers of at least one fatty acid comprising at least 16 carbon atoms.

96. The anhydrous composition according to claim 95, wherein said at least one fatty acid is chosen from oleic acid, linoleic acid and linolenic acid.

97. The anhydrous composition according to one of claims 93 to 96, wherein said diamines are chosen from ethylenediamine, hexylenediamine, hexamethylenediamine, and phenylenediamine and said triamines are chosen from ethylenetriamine.

98. The anhydrous composition according to one of claims 92 to 97, wherein said at least one polyamide polymer is chosen from polymers comprising at least one terminal carboxylic acid group.

99. The anhydrous composition according to claim 98, wherein said at least one terminal carboxylic acid group is esterified with at least one alcohol chosen from monoalcohols comprising at least 4 carbon atoms.

100. The anhydrous composition according to one of claims 92 to 99, wherein said at least one organogelator and/or said at least one structuring polymer have an affinity with a chemical portion of one of the oils forming the liquid fatty phase of the composition so that hydrogen bonds with the oils are formed.

101. The anhydrous composition according to one of claims 92 to 100, further comprising at least one amphiphilic compound that is liquid and non-volatile at room temperature and has a hydrophilic/lipophilic balance value of less than 12.

102. The anhydrous composition according to claim 101, wherein said at least one amphiphilic compound comprises a lipophilic part linked to a polar part, the lipophilic part comprising a carbon-based chain comprising at least 8 carbon atoms.

103. The anhydrous composition according to one of claims 92 to 102, wherein said at least one liquid fatty phase of the composition, further comprises at least one oil.

104. The anhydrous composition according to one of claims 92 to 103, further comprising at least one additional rheological agent.

105. The anhydrous composition according to claim 104, wherein said at least one additional rheological agent is chosen from waxes, polymeric gelling agents and mineral gelling agents for the liquid fatty phase.

106. The anhydrous composition according to one of claims 92 to 105, further comprising at least one coloring agent.

107. The anhydrous composition according to one of claims 92 to 116, wherein said composition is in the form of a rigid gel.

108. The anhydrous composition according to one of claims 92 to 107, wherein said composition is in the form of an anhydrous stick.

109. The composition according to one of claims 1 to 108, wherein said composition further comprises at least one additional Theological agent, wherein said at least one additional rheological agent is hydrophobic-treated fumed silica.

110. The composition according to one of claims 92 to 109, wherein said composition further comprises at least one wax.

111. A mascara, an eyeliner, a foundation, a lipstick, a blusher, a make-up-removing product, a make-up product for the body, a nail composition, an eyeshadow, a face powder, a concealer product, a shampoo, a conditioner, an antisun product or a care product for the lips, hair or nails comprising a composition comprising at least one liquid fatty phase in said mascara, eyeliner, foundation, lipstick, blusher, make-up-removing product, make-up product for the body, nail composition, eyeshadow, face powder, concealer product, shampoo, conditioner, antisun product or care product for the lips, hair or nails, which comprises:
(i) at least one structuring polymer comprising:
a polymer skeleton which comprises at least one hydrocarbon-based repeating unit comprising at least one hetero atom; and
(ii) at least one organogelator.

112. A deodorant product or a care product for the skin or body comprising an anhydrous composition comprising at least one liquid fatty phase in said product which comprises:
(i) at least one structuring polymer comprising:
a polymer skeleton which comprises at least one hydrocarbon-based repeating unit comprising at least one hetero atom; and
(ii) at least one organogelator.

113. A care and/or treatment and/or make-up composition for keratin materials comprising an anhydrous composition comprising at least one liquid fatty phase which comprises:
(i) at least one structuring polymer comprising:
a polymer skeleton which comprises at least one hydrocarbon-based repeating unit comprising at least one hetero atom; and
(ii) at least one organogelator.

114. A care and/or treatment and/or make-up composition for keratinous fibers, lips or skin comprising at least one liquid fatty phase in said care and/or treatment and/or make-up composition for keratinous fibers, lips or skin which comprises:
(i) at least one structuring polymer comprising:
a polymer skeleton which comprises at least one hydrocarbon-based repeating unit comprising at least one hetero atom; and
(ii) at least one organogelator.

115. A lipstick composition in stick form comprising at least one continuous liquid fatty phase, at least one organogelator for the fatty phase and at least one non-waxy structuring polymer having a weight-average molecular mass of less than 100 000, said continuous liquid fatty phase, said at least one organogelator for the fatty phase and said at least one non-waxy structuring polymer being present in said lipstick composition.

116. A method for care, make-up or treatment of keratin materials comprising applying to said keratin materials an anhydrous composition comprising at least one liquid fatty phase which comprises:
(i) at least one structuring polymer comprising:
a polymer skeleton which comprises at least one hydrocarbon-based repeating unit comprising at least one hetero atom; and
(ii) at least one organogelator.

117. A method for care, make-up or treatment of keratinous fibers, lips, or skin comprising applying to said keratinous fibers, lips, or skin a composition comprising at least one liquid fatty phase which comprises:
(i) at least one structuring polymer comprising:
a polymer skeleton which comprises at least one hydrocarbon-based repeating unit comprising at least one hetero atom; and
(ii) at least one organogelator.

118. A method for providing an anhydrous composition having at least one property chosen from a solid appearance, non-exudation, shear-strength, gloss, and comfortable deposit on keratin materials chosen from lips, skin, and keratinous fibers, comprising including in said composition at least one liquid fatty phase which comprises:
(i) at least one structuring polymer comprising:
a polymer skeleton which comprises at least one hydrocarbon-based repeating unit comprising at least one hetero atom; and
(ii) at least one organogelator.

119. An anhydrous composition comprising at least one liquid fatty phase which comprises:
(i) at least one structuring polymer comprising:
a polymer skeleton which comprises at least one hydrocarbon-based repeating unit comprising at least one hetero atom; and
(ii) at least one organogelator,
wherein said at least one structuring polymer is a compound of formula XVII:
R—O—CO—NH—R′—NH—CO—NH—R″—NH—CO—NH—R′—NH—CO—OR
wherein R represents CₙH₂ₙ₊₁— or CₘH₂ₘ₊₁ (CₚH₂ₚO)ᵣ—; n represents an integer having a value of from 4 to 22; m represents an integer having a value of from 1 to 18; p represents an integer having a value of from 2 to 4; and r represents an integer having a value of from 1 to 10;
R′ represents: 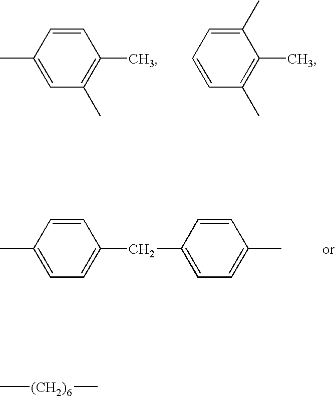
or —(CH₂)₆—
and R″ represents:

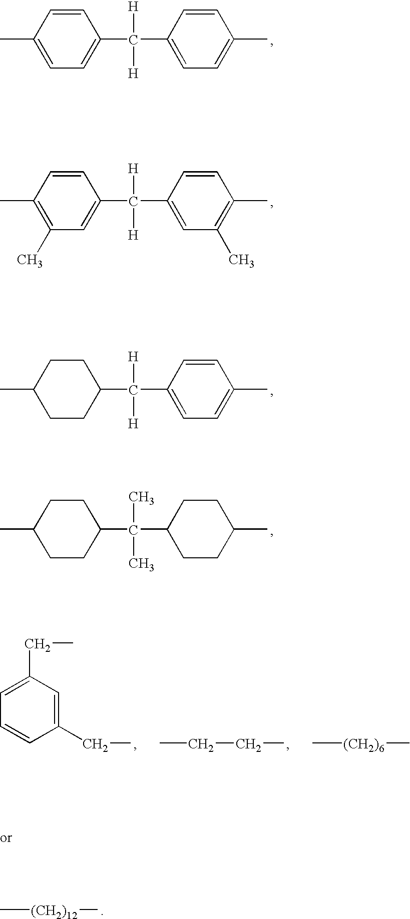

120. A structured composition comprising at least one liquid fatty phase structured with at least one structuring polymer comprising a polymer skeleton comprising at least one hydrocarbon-based repeating unit comprising at least one hetero atom, wherein the at least one structuring p60olymer further comprises at least one chain chosen from
(i) terminal fatty chains, optionally functionalized, chosen from alkyl and alkenyl chains, bonded to the polymer skeleton via at least one linking group chosen from amides, ureas, and esters, and
(ii) pendant fatty chains, optionally functionalized, chosen from alkyl and alkenyl chains, bonded to the polymer skeleton via at least one linking group chosen from amides, ureas, and esters,
wherein when said at least one linking group is chosen from esters, said at least one terminal fatty chain is chosen from branched alkyl groups, and further comprising at least one organogelator.

121. A make up or care or treatment composition for the skin, the lips, or keratinous fibers comprising a structured composition comprising at least one liquid fatty phase structured with at least one structuring polymer comprising a polymer skeleton comprising at least one hydrocarbon-based repeating unit comprising at least one hetero atom, at least one organogelator able to gel the liquid fatty phase, and at least one coloring agent.

122. A method of making up or caring for skin, lips, or keratinous fibers comprising applying to said skin, lips, or keratinous fibers a structured composition comprising at least one liquid fatty phase structured with at least one structuring polymer comprising a polymer skeleton comprising at least one hydrocarbon-based repeating unit comprising at least one hetero atom and at least one organogelator able to gel the liquid fatty phase.

123. An anhydrous composition according to one of claims 1 to 118, wherein said at least three hydrocarbon-based repeating units are identical.

124. A composition comprising at least one liquid fatty phase which comprises:
(i) at least one structuring polymer chosen from urea urethanes having the following formula XVI:
R—O—CO—NH—R′—NH—CO—NH—R″—NH—CO—NH—R′—NH—CO—OR (XVI)
wherein R represents CₙH₂ₙ₊₁—, wherein n represents an integer having a value greater than 22 or CₘH₂ₘ₊₁ (OCₚH₂ₚ)ᵣ—, wherein m represents an integer having a value of greater than 18, p represents an integer having a value of from 2 to 4, and r represents an integer having a value of from 1 to 10.
R′ represents: 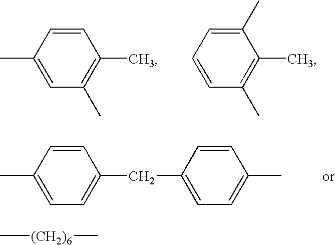
or —(CH₂)₆—
and R″ represents: 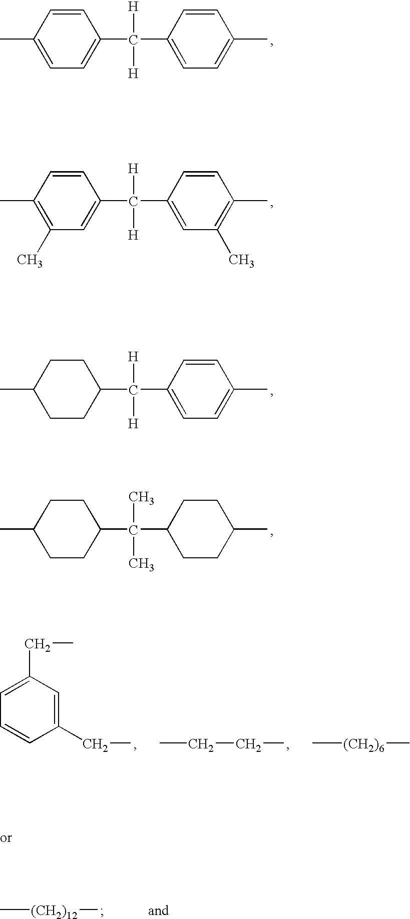
(ii) at least one organogelator.

125. A composition comprising at least one liquid fatty phase which comprises:
(i) at least one structuring polymer comprising
a polymer skeleton which comprises at least one hydrocarbon-based repeating unit comprising at least one hetero atom with the proviso that said at least one hetero atom is not nitrogen; and
(ii) at least one organogelator.

126. A composition comprising at least one liquid fatty phase which comprises:
(i) at least one structuring polymer comprising:
a polymer skeleton which comprises a) at least one hydrocarbon-based repeating unit comprising at least one hetero atom and b) at least one of:
at least one terminal fatty chain, optionally functionalized, chosen from alkyl chains and alkenyl chains, wherein said at least one terminal fatty chain is bonded to said polymer skeleton via at least one linking group; and
at least one pendant fatty chain, optionally functionalized, chosen from alkyl chains and alkenyl chains, wherein said at least one pendant fatty chain is bonded to said polymer skeleton via at least one linking group; and
(ii) at least one organogelator.
